(19) United States
(12) Patent Application Publication
(10) Pub. No.: US 2019/0000564 A1
(43) Pub. Date: Jan. 3, 2019

(54) SYSTEM AND METHOD FOR MEDICAL IMAGING

(71) Applicant: The Johns Hopkins University, Baltimore, MD (US)

(72) Inventors: Nassir Navab, Baltimore, MD (US); Bernhard Fuerst, Baltimore, MD (US); Mohammadjavad Fotouhighazvini, Baltimore, MD (US); Sing Chun Lee, Baltimore, MD (US)

(21) Appl. No.: 16/067,572

(22) PCT Filed: Dec. 30, 2016
(86) PCT No.: PCT/US16/69458

§ 371 Date: Jun. 29, 2018

Related U.S. Application Data
(60) Provisional application No. 62/273,229, filed on Dec. 30, 2015.

(51) Int. Cl. A61B 34/20; A61B 6/00; A61B 6/12; A61B 5/00; H04N 13/254; G06T 7/521; G06T 7/80; A61B 34/30

(52) U.S. Cl. CPC A61B 34/20; A61B 2090/3764; A61B 6/5241; A61B 6/5247; A61B 6/12; A61B 5/0077; H04N 13/254; G06T 7/521; G06T 7/80; A61B 34/30; A61B 6/582; A61B 6/505; G06T 2207/10024; G06T 2207/10081; G06T 2207/30004; A61B 6/4085

(57) ABSTRACT

The present disclosure provides a system and method for generating medical images. The method utilizes a novel algorithm to co-register Cone-Beam Computed Tomography (CBCT) volumes and additional imaging modalities, such as optical or RGB-D images.

### CROSS REFERENCE TO RELATED APPLICATION(S)

This application claims the benefit of priority under 35 U.S.C. § 119(e) of U.S. Ser. No. 62/273,229, filed Dec. 30, 2015, the entire contents of which is incorporated herein by reference in its entirety.

### BACKGROUND OF THE INVENTION

### Field of the Invention

The invention relates generally to image processing and more particularly to co-registration of images from different imaging modalities.

### Background Information

Cone-Beam Computed Tomography (CBCT) is one of the primary imaging modalities in radiation therapy, dentistry, and orthopedic interventions. While providing crucial intraoperative imaging, CBCT is bounded by its limited imaging volume, motivating the use of image stitching techniques. Current methods rely on overlapping volumes, leading to an excessive amount of radiation exposure, or on external tracking hardware, which may increase the setup complexity.

CBCT apparatuses are known in the art and provide tomographic images of an anatomic portion by acquiring a sequence of bi-dimensional radiographic images during the rotation of a system that comprises an X-ray source and an X-ray detector around the anatomic part to be imaged.

A CBCT apparatus typically includes: an X-ray source projecting a conic X-ray beam (unless it is subsequently collimated) through an object to be acquired; a bi-dimensional X-ray detector positioned so as to measure the intensity of radiation after passing through the object; a mechanical support on which said X-ray source and detector are fixed, typically called a C-arm; a mechanical system allowing the rotation and the translation of said support around the object, so as to acquire radiographic images from different positions; an electronic system adapted to regulate and synchronize the functioning of the various components of the apparatus; and a computer or similar, adapted to allow the operator to control the functions of the apparatus, and to reconstruct and visualize the acquired images.

The name of the C-arm is derived from the C-shaped arm used to connect the X-ray source and X-ray detector to one another. There are substantially two kinds of such apparatuses on the market: a first kind where the patient stands or sits vertically during the acquisition; and a second kind where the patient lies on a table.

A need exists for an improved method and system for generating medical images which reduce radiation exposure and/or reduce the need of external hardware and system complexity in a medical environment.

### SUMMARY OF THE INVENTION

The invention provides a system and method utilizing an innovative algorithm to co-register CBCT volumes and additional imaging modalities.

In one aspect, the invention provides a medical imaging apparatus. The apparatus includes: a) a Cone-Beam Computed Tomography (CBCT) imaging modality having an X-ray source and an X-ray detector configured to generate a series of image data for generation of a series of volumetric images, each image covering an anatomic area; b) an auxiliary imaging modality configured to generate a series of auxiliary images; and c) a processor having instructions to generate a global volumetric image based on the volumetric images and the auxiliary images. The processor is configured to perform an image registration process including co-registering the volumetric images and the auxiliary images, wherein co-registration includes stitching of non-overlapping volumetric images to generate the global volumetric image. In embodiments, the auxiliary imaging modality is an optical imaging modality configured to generate optical images or a depth imaging modality, such as an RGB-D camera. In embodiments, the imaging modalities are housed in a C-arm device.

In another aspect, the invention provides a method for generating an image, such as a medical image. The method includes: a) generating a series of image data using a Cone-Beam Computed Tomography (CBCT) imaging modality for generation of a series of volumetric images, each image covering an anatomic area; b) generating a series of auxiliary images using an auxiliary imaging modality; and c) generating a global volumetric image based on the volumetric images and the auxiliary images, thereby generating an image. The global volumetric image is generated via an image registration process including co-registering the volumetric images and the auxiliary images, wherein co-registration includes stitching of non-overlapping volumetric images to generate the global volumetric image. In embodiments, the auxiliary imaging modality is an optical imaging modality configured to generate optical images or a depth imaging modality, such as an RGB-D camera. In embodiments, the imaging modalities are housed in a C-arm device.

In another aspect, the invention provides a medical robotic system. The system includes a memory for receiving an image generated via the method of the invention; and a processor configured for at least semi-automatically controlling the medical robotic system based on the received image.

In another aspect, the invention provides a method of performing a medical procedure utilizing the system and/or the imaging methodology of the present invention.

In another aspect, the invention provides methodology which utilizes mathematical algorithms to calibrate a system of the invention and utilize the system to image and/or track an object.

### BRIEF DESCRIPTION OF THE DRAWINGS

FIG. 1 is a schematic diagram of a broken femur. The 3D misalignment of bones may be difficult to quantify using 2D images. CBCT contributes as a valuable tool for interventions in which the 3D alignment is of importance, for instance in acute fracture treatment or joint replacement.

FIG. 2 is a schematic showing a system in one embodiment of the disclosure. A mobile C-arm, the positioning-laser, and an optical camera are illustrated. The mirror aligns the optical camera and X-ray source centers. The patient motion relative to the C-arm is estimated by observing both the positioning-laser and natural features on the patient's surface. The 3D positions of the features are estimated using the depth of the nearest positioning-laser on the patient, of which the depth is based on calibration.

FIG. 3 is an image which shows the overlay of two frames to illustrate the feature correspondences to estimate the movement of a patient. From both frames, the positioning-laser and natural surface features are extracted. The tracking results of the matched features in frame k (+) and frame k+1 (o) are illustrated as yellow lines.

FIGS. 4(a)-4(d) are images illustrating a method of the invention. Absolute distance of the aligned sub-volumes in 4(a) is measured (415.37 mm), and compared to the real world measurements (415 mm) of the femur phantom in 4(b). Similarly, a fiducial phantom was scanned and the vision-based stitching 4(c) compared to the real world object 4(d). For visualization purposes and volumetric appearance in 4(a) and 4(c), multiple parallel slices are averaged.

FIG. 5 is a graph showing experimental results utilizing a system in one embodiment of the disclosure. The plot illustrates duration of the intervention, number of X-ray images taken, radiation dose, K-wire placement error, and surgical task load, where each bar shows the accumulated values using one of the systems (conventional X-ray, RGB/X-ray fusion, or RBGD/DRR). Each measure is normalized relative to the maximum value observed. The ‘*’ symbols indicate significant differences.

FIG. 6 is a schematic diagram relating to the offline calibration of RGBD camera to CBCT origin is per-formed by introducing an arbitrary object into the common views of both devices. Before an intervention begins, CBCT and surface scans of the patient are acquired simultaneously. During the intervention, the fused visualization of patient's surface, surgeon's hands and tools, together with simulated X-ray images (DRR), are displayed to assist the surgeon

FIG. 7 is a schematic diagram showing system setup. A depth camera is rigidly mounted on the detector, so that the field of view and depth of view cover the CBCT volume.

FIG. 8 is a schematic diagram showing a checkerboard designed to be fully visible in both the RGB and the X-ray image.

FIG. 9 is a schematic diagram. The relative displacement of the CBCT volume (CBCT′TCBCT) can be estimated using the tracking data computed using the camera mounted on the C-arm. This requires the calibration of camera and X-ray source (XTRGB), and the know relationship of X-ray source and CBCT volume (CBCTTX). The pose of the marker is observed by the camera (RGBTM), while the transformation from marker pose to CBCT volume (CBCTTM) is computed once and assumed to remain constant.

FIG. 10 is a schematic diagram. An infrared tracking system is used for alignment and stitching of CBCT volumes and provides a baseline for the evaluation of vision-based techniques. The necessity of tracking both the C-arm and patient causes an accumulation of errors, while also reducing the work space in the OR by introducing additional hardware.

FIG. 11 is a graphical representation showing data relating to reprojection error of X-ray to RGB.

FIG. 12 is a graphical representation showing data relating to reprojection error of RGB to IR

FIG. 13 is a schematic showing workflow of the tracking of surgical tools for interventional guidance in the mixed reality environment. The system is pre-calibrated which enables a mixed reality visualization platform. During intervention, surgeon first selects the tool model and defines the trajectory (planning) on the medical data. Next, the mixed reality environment is used together with the tracking outcome for supporting the tool placement.

### DETAILED DESCRIPTION OF THE INVENTION

In the following detailed description of the embodiments of the invention, numerous specific details are set forth in order to provide a thorough understanding of the disclosed embodiments. However, it will be obvious to one skilled in the art that the embodiments of this disclosure may be practiced without these specific details. In other instances well known methods, procedures, components, and circuits have not been described in detail so as not to unnecessarily obscure aspects of the embodiments of the invention.

The embodiments below describe various instruments and portions of instruments in terms of their state in three-dimensional space. As used herein, the term “position” refers to the location of an object or a portion of an object in a three-dimensional space (e.g., three degrees of translational freedom along Cartesian X,Y,Z coordinates). As used herein, the term “orientation” refers to the rotational placement of an object or a portion of an object (three degrees of rotational freedom—e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom). As used herein, the term “shape” refers to a set of poses, positions, or orientations measured along an object.

Before the present systems and methods are described, it is to be understood that this invention is not limited to particular systems, methods, and experimental conditions described, as such systems, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

Cone-Beam Computed Tomography (CBCT) is one of the primary imaging modalities in radiation therapy, dentistry, and orthopedic interventions. While providing crucial intraoperative imaging, CBCT is bounded by its limited imaging volume, motivating the use of image stitching techniques. Current methods rely on overlapping volumes, leading to an excessive amount of radiation exposure, or on external tracking hardware, which may increase the setup complexity. The present invention utilizes and an optical camera attached to a CBCT enabled C-arm, and co-register the video and X-ray views. An algorithm recovers the spatial alignment of non-overlapping CBCT volumes based on the observed optical views, as well as the laser projection provided by the X-ray system. First, the inventors estimate the transformation between two volumes by automatic detection and matching of natural surface features during the patient motion. Then, 3D information is recovered by reconstructing the projection of the positioning-laser onto an unknown curved surface, which enables the estimation of the unknown scale.

In embodiments, a RGB-D or depth camera can be used to reconstruct the patient's surface. This allows the computation of the patient's movement relative to the depth camera. If the depth camera is calibrated to the CBCT volume, a fusion of surface and CT volume is possible, enabling 3D/3D visualization (for instance arbitrarily defined views of patient surface and x-rays) or intuitive tracking of tools.

The present invention is crucial in next generation operating rooms, enabling physicians to target points on bone (for k-wire insertion), target areas for biopsies (both soft and boney tissue), or intuitively visualized foreign bodies in 3D.

In this respect, Examples 1 and 4 herein set forth the system and methodology in embodiments of the invention. As discussed in Examples 1 and 4, the CBCT-enabled motorized C-arm is positioned relative to the patient by utilizing the positioning-lasers, which are built into the image intensifier and C-arm base. To enable the stitching of multiple sub-volumes, the transformation of the patient relative to the C-arm center must be recovered. In contrast to existing techniques the present technique does not require additional hardware setup around the C-arm, but a camera is attached to the C-arm in such manner that it does not obstruct the surgeons access to the patient. By using one mirror, the camera and the X-ray source centers are optically identical. The system setup is outlined in FIG. 2 in one embodiment.

The proposed technique is an overlap-independent, low dose, and accurate stitching method for CBCT sub-volumes with minimal increase of workflow complexity. An optical camera is attached to a mobile C-arm, and used the positioning laser to recover the 3D depth scales, and consequently aligned the sub-volumes. As a result of this method, the stitching is performed with low dose radiation, linearly proportional to the size of non-overlapping sub-volumes. It is expected that this is applicable to intraoperative planning and validation for long bone fracture or joint replacement interventions, where multi-axis alignment and absolute distances are difficult to visualize and measure from the 2D X-ray views. The approach does not limit the working space, nor does it require any additional hardware besides a simple camera. The C-arm remains mobile and independent of the OR. One requirement is that the C-arm does not move during the CBCT acquisition, but the inventors believe that the use of external markers could solve this problem and may yield a higher accuracy. However, in our scenario the inventors intentionally did not rely on markers, as they would increase complexity and alter the surgical workflow. The approach uses frame-to-frame tracking, which can cause drift. In fact, the ICP verification helps us to detect such drifts as it is based on points which were not used for motion estimation. Therefore, if the estimated motion from ICP increases over time, we can detect the drift and use ICP to correct if necessary. Alternatively, the transformations could be refined using bundle adjustments. Further studies on the effectiveness during interventions are underway. Also, the reconstruction of the patient surface during the CBCT acquisition may assist during the tracking of the patient motion.

As discussed above, 3D visualization of the underlying medical anatomy is crucial during several orthopedics interventions. Bony structures could be visualized in 3D by acquiring CBCT scans. However, the field of view of a CBCT is very limited, and several acquisitions are required to observe larger anatomies.

As shown in the embodiments of Examples 1 and 4, the geometric displacement (transformation) of the C-arm relative to the patient is computed between two (or multiple) CBCT acquisitions. This transformation is used to compute the relative pose of the two scans, hence allowing us to stitch the non-overlapping CBCT volumes and construct larger volumes. The geometric transformations are computed using visual information from a color camera attached to the C-arm source.

To stitch multiple non-overlapping CBCT volumes, we use a camera which is rigidly attached to the C-arm and co-calibrated with the X-ray source. This system does not use any additional markers, and only relies on visual information from a camera attached to a CBCT enabled C-arm.

To recover 3D information from a single 2D camera on the C-arm, we use the guidance-laser attached to the base of the C-arm. Before recovering the scale of the color information, a camera to laser plane calibration is required. This is done by intersecting a checkerboard pattern with the laser line at several poses. Then, we project the laser on the patient, and use the known information from the laser plane to recover the scale of the visual features.

The C-arm remains self-contained and flexible in this embodiment, where both the patient (surgical bed) and the C-arm can be displaced.

In another embodiment of the present invention described in Example 3, the proposed technique uses an RGBD camera mounted on a mobile C-arm, and recovers a 3D rigid-body transformation from the RGBD surface point clouds to CBCT. The transformation is recovered using Iterative Closest Point (ICP) with a Fast Point Feature Histogram (FPFH) for initialization. The general workflow is illustrated in Example 3 and is comprised of an offline calibration, patient data acquisition and processing, and intra-operative 3D augmented reality visualization. Example 3 describes the system setup, calibration phantom characteristics, transformation estimation, and the augmented reality overlay.

As discussed above, X-Ray images are the crucial intra-operative tool for orthopedic surgeons to understand the anatomy for their k-wire/screw placements. 2D images lack of 3D information results in difficult mental alignment for entry point localization, thus lead to multiple failure attempts, lengthy operation time and team frustration.

The solution provided in Example 3 is a 3D mixed reality visualization system provided by a light-weight rigidly mounted RGBD camera, which is calibrated to the CBCT space. In this embodiment, the RGBD camera is rigidly mounted near the C-arm detector. A one-time calibration is perform to recover the spatial relationship between RGBD camera space and CBCT space. During the intervention, intra-operative CBCT is scanned and patient surface is captured and reconstructed simultaneously by the RGBD camera. After the scan, a mixed reality sense can be generated with DRR generated from the CBCT data, reconstructed patient surface and a live feedback point clouds of hands/surgical tools.

In the embodiment of Example 3, the system integrates an RGBD camera into mobile C-arm. The camera is rigidly mounted near the C-arm detector, and thus only requires one-time calibration to recover the spatial relationship to the CBCT space. A reliable calibration phantom and algorithm are described for this calibration process. The calibration algorithm works for any arbitrary objects that has non rotational symmetric shape and visible in both CBCT and RGBD spaces. It is evaluated in terms of repeatability, accuracy, invariant to noise and shapes. Provided the calibration result, a 3D mixed realty visualization can be generated, which allows orthopedic surgeons understand the surgical sense in a more intuitive and faster way. It helps to shorten the operation time, reduce radiation and team feel less frustration. The mixed reality visualization can provide multiple sense at any arbitrary angles, which even allows surgeon to look through the anatomy at an angle that is not possible to acquire in real life.

As discussed in Example 3, the present invention provides a methodology to calibrate a RGBD camera rigidly mounted on a C-arm and a CBCT volume. This combination enables intuitive intra-operative augmented reality visualization. The inventors evaluated the accuracy and robustness of the algorithm using several tests. Although the spatial resolution of the RGBD cameras in depth is poor (approximately +−5% of the depth), the inventors achieve a reasonable registration accuracy of 2.58 mm. The inventors have presented two applications with high clinical impact. First, image-guided drilling for cannulated sacral screw placement was demonstrated. Finally, the inventors concluded the experiments with a simulated foreign body removal using shrapnel models. To achieve the fused RGBD and DRR view, multiple steps are required. First, the CBCT and patient's surface scans are acquired. The FPFH matching for fast initialization of ICP yields a robust and efficient calibration of data extracted from CBCT and RGBD. This enables the data overlay, resulting in an augmented reality scene. The calibration accuracy is strongly dependent on the quality of the depth information acquired from the RGBD camera. Even though the cameras used in this paper provide a limited depth accuracy, we could show that our calibration technique is robust.

In contrast to other calibration techniques, a pre-defined marker or known 3D structure is not required. Theoretically, the calibration technique functions with any arbitrary object for which the surface is visible in the CBCT volume and yields enough structural features. In a clinical scenario, a system constructed as our design would require a one-time calibration or at the discretion of the user.

The fusion of CBCT and RGBD into one common coordinate space enables several new concepts. First, any arbitrary view can be visualized as the spatial restrictions in terms of C-arm placement no longer apply. For instance, a view along the spine can be visualized while placing a Jamshidi needle. Secondly, the augmented reality scene can be viewed from different view point simultaneously. This enables surgeons to align tools in all dimensions at the same time, possibly saving significant OR time.

In the embodiment described in Example 3, changes in the environment are not tracked. For instance, moving the surgical table or RGBD camera may result in the loss of proper image alignment, which motivates further development of the CBCT and RGBD system. Beyond the aforementioned possibilities, the fusion of RGBD and CBCT could facilitate intra-operative surgical navigation as the RGBD camera could be used for tool or activity tracking. Understanding the activity would enable the automatic adjustment of the view in order to provide the most optimal view during interventions. The proposed technique contributes to a robust calibration for RGBD and CBCT data, and enables an entire new field of novel applications for image-guided interventions.

As discussed in Example 2, another embodiment of the invention is set forth. The inventors design and perform a usability study to compare the performance of surgeons and their task load using three different mixed reality systems during K-wire placements. The three systems are interventional X-ray imaging, X-ray augmentation on 2D video, and 3D surface reconstruction augmented by digitally reconstructed radiographs and live tool visualization.

C-arm fluoroscopy is the crucial imaging modality in several orthopedic and trauma interventions. The main challenge in these procedures is the matching of X-ray images acquired using a C-arm with the medical instruments and the patient. This dramatically increases the complexity of pelvic surgeries.

Example 2 sets forth a 3D augmented reality environment that fuses real-time 3D information from an RGBD camera attached to the C-arm, with simulated X-ray images, so-called Digitally Reconstructed Radiographs (DRRs), from several arbitrary perspectives. A pre-clinical study is designed to evaluate the efficiency and benefits of this system in placing K-wires in pelvic fractures.

As discussed in detail in Example 2, a 3D Augmented Reality (AR) environment is used for placing K-wires inside the bone using dry phantoms. To this end, we conducted 8 pre-clinical user studies, where several surgical efficiency measures such as duration, number of X-ray images, cumulative area dose, and accuracy of the wire placement are identified and evaluated using the 3D surgical AR visualization. In addition, using the surgical task load index the system is compared to the standard fluoro-based procedure, as well as 2D AR visualization.

In Example 2, the inventors first describe the imaging systems to be compared. These include conventional intra-operative X-ray imaging, X-ray image augmented 2D video, and a novel 3D RGBD view augmented with DRR. Finally, the inventors present the questionnaires and statistical methods to perform the usability study.

In Example 2 the inventors presented a thorough usability study using three different mixed reality visualization systems to perform K-wire placement into the superior pubic ramus. This procedure was chosen because of the high clinical relevance, frequent prevalence, and the especially challenging minimal invasive surgical technique. Attention was focused on the usability and clinical impact of the three different visualization systems. For that reason we were not only interested in the quality of a procedure (e.g. accuracy), but also in the workload and frustration that the surgeons experienced while using the different systems. 21 interventions were observed performed by 7 surgeons, and used the Surgical TLX to evaluate the task load.

The results show that the 3D visualization yields the most benefit in terms of surgical duration, number of X-ray images taken, overall radiation dose and surgical workload. Results indicate that the 3D AR visualization leads to significantly improved visualization, and confirms the importance and effectiveness of this system in reducing the radiation exposure, surgical duration, and effort and frustration for the surgical team.

As discussed in Example 5, another embodiment of the invention is set forth. Navigation during orthopedics interventions greatly help the surgeons for entry point localization and thus reduces the use of X-Ray images. However, additional setup and hardware are often required for an accurate navigation system. In addition, more often navigation systems are over-engineered for accuracy, and thus the change of workflow is disregarded. In reality, an easy-to-integrate guidance system that brings the surgeon to a better starting point is sufficient to improve the accuracy, shorten the operation time and reduce radiation dose.

Instead of additional tracking system for the navigation, Example 5 discusses an embodiment of the invention which includes use of a light-weight RGBD camera and novel depth camera based tracking algorithm to provide a guidance system for orthopedics surgery. A calibrated RGBD camera is attached to a mobile C-arm. This camera provides the live depth camera view, which is then used by the novel tracking algorithm to track the tool and visualize it with planned trajectory for guidance.

The system makes use of a calibrated RGBD camera that is rigidly mounted near the C-arm detector. This camera provides the live depth camera view, which is then be used for simultaneous sense reconstruction, object recognition, and tool tracking.

The tracking algorithm is model based and it computes the live 3D features from the depth images. The 3D features are then used to recreate the sense and segment objects from the sense. Finally, the segmented objects are compared to the model for tool tracking. The tracking result is then applied to the mixed reality visualization sense to give a tracked surgical model with projected drilling direction, which can then be visually compared to the planned trajectory, and hence intuitively guide the surgeon where is a good starting point. In embodiments, additional visualization depth cue can be applied to further improve the depth perception and sense understanding, which further help surgeons to quickly set up their entry points.

The present invention is described partly in terms of functional components and various processing steps. Such functional components and processing steps may be realized by any number of components, operations and techniques configured to perform the specified functions and achieve the various results. For example, the present invention may employ various materials, computers, data sources, storage systems and media, information gathering techniques and processes, data processing criteria, algorithmic analyses and the like, which may carry out a variety of functions. In addition, although the invention is described in the medical context, the present invention may be practiced in conjunction with any number of applications, environments and data analyses; the systems described herein are merely exemplary applications for the invention.

Methods according to various aspects of the present invention may be implemented in any suitable manner, for example using a computer program operating on or in connection with the system. An exemplary system, according to various aspects of the present invention, may be implemented in conjunction with a computer system, for example a conventional computer system comprising a processor and a random access memory, such as a remotely-accessible application server, network server, personal computer or workstation. The computer system also suitably includes additional memory devices or information storage systems, such as a mass storage system and a user interface, for example a conventional monitor, keyboard and tracking device. The computer system may, however, comprise any suitable computer system and associated equipment and may be configured in any suitable manner. In one embodiment, the computer system comprises a stand-alone system. In another embodiment, the computer system is part of a network of computers including a server and a database.

The software required for receiving, processing, and analyzing data may be implemented in a single device or implemented in a plurality of devices. The software may be accessible via a network such that storage and processing of information takes place remotely with respect to users.

The system may also provide various additional modules and/or individual functions. For example, the system may also include a reporting function, for example to provide information relating to the processing and analysis functions. The system may also provide various administrative and management functions, such as controlling access and performing other administrative functions.

The following examples are provided to further illustrate the advantages and features of the present invention, but are not intended to limit the scope of the invention. While they are typical of those that might be used, other procedures, methodologies, or techniques known to those skilled in the art may alternatively be used.

### Example 1

### Vision-Based Intraoperative Cone-Beam CT Stitching for Non-Overlapping Volumes

Cone-Beam Computed Tomography (CBCT) is one of the primary imaging modalities in radiation therapy, dentistry, and orthopedic interventions. While providing crucial intraoperative imaging, CBCT is bounded by its limited imaging volume, motivating the use of image stitching techniques. Current methods rely on overlapping volumes, leading to an excessive amount of radiation exposure, or on external tracking hardware, which may increase the setup complexity. We attach an optical camera to a CBCT enabled C-arm, and co-register the video and X-ray views. Our novel algorithm recovers the spatial alignment of non-overlapping CBCT volumes based on the observed optical views, as well as the laser projection provided by the X-ray system. First, we estimate the transformation between two volumes by automatic detection and matching of natural surface features during the patient motion. Then, we recover 3D information by reconstructing the projection of the positioning-laser onto an unknown curved surface, which enables the estimation of the unknown scale. We present a full evaluation of the methodology, by comparing vision- and registration-based stitching.

Cone-Beam Computed Tomography (CBCT) enables intraoperative 3D imaging for various applications, for instance orthopedics, dentistry or radiation therapy. Consequently, CBCT is aimed at improving localization, structure identification, visualization, and patient positioning. However, the effectiveness of CBCT in orthopedic surgeries is bounded by its limited field of view, resulting in small volumes. Intraoperative surgical planning and verification could benefit of an extended field of view. Long bone fracture surgeries could be facilitated by 3D absolute measurements and multi-axis alignment in the presence of large volumes, assisting the surgeon's mental alignment.

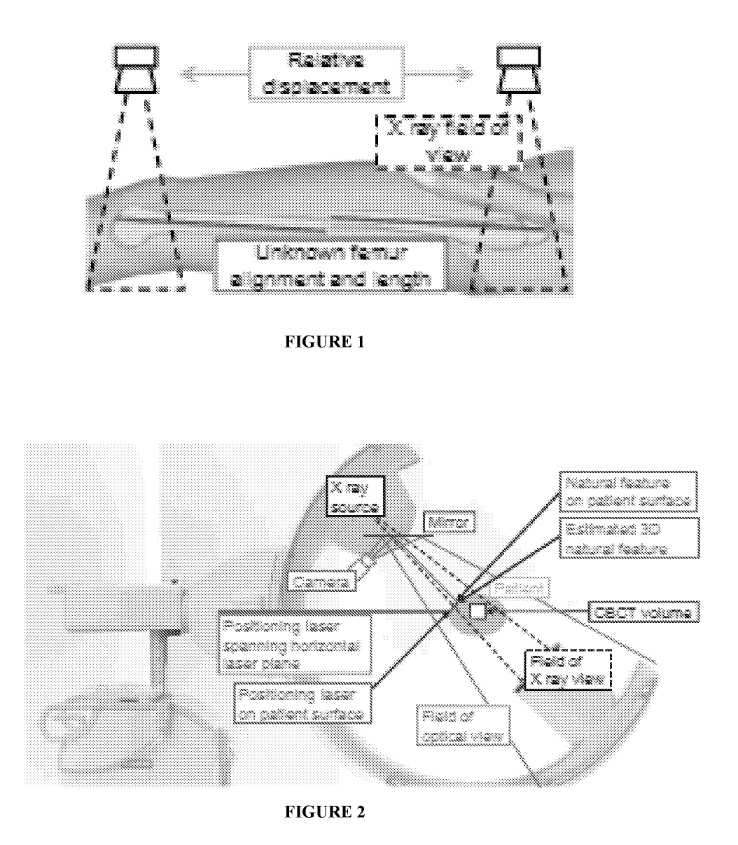

The value of stitched fluoroscopy images for orthopedic surgery was previously investigated. Radio-opaque referencing markers attached to the tool were used to perform the stitching. Trajectory visualization and total length measurement were the most frequent features used by the surgeons in the stitched view. The outcome was overall promising for future development, and the usability was counted as good. Similarly, prior studies employed X-ray translucent references positioned under the bone for 2D X-ray mosaicing. In experiments, optical features acquired from an adjacent camera were used to recover the transformation. The aforementioned methods all benefit from external features for 2D mosaicing, thus do not require large overlaps. However, it remains a challenge to generalize these approaches to perform 3D volume stitching, as illustrated in FIG. 1.

A validation study on using 3D rotational X-ray over conventional 2D X-rays was conducted for intra-articular fractures of the foot, wrist, elbow, and shoulder. The outcome reported a reduction of indications for revision surgery. A panoramic CBCT was proposed by stitching overlapping X-rays acquired from all the views around the interest organ. Reconstruction quality is ensured by introducing a sufficient amount of overlapping regions, which in return increases the X-ray dose. Moreover, the reconstructed volume is vulnerable to artifacts introduced by image stitching. An automatic 3D image stitching technique was previously proposed. Under the assumption that the orientational misalignment is negligible, and sub-volumes are only translated, the stitching is performed using phase correlation as a global similarity measure, and normalized cross correlation as the local cost. Sufficient overlaps are required to support this method. To reduce the X-ray exposure, prior knowledge from statistical shape models was incorporated to perform a 3D reconstruction.

Previous approaches are either limited to the overlap size or the existing prior shape models. Providing large overlaps will significantly increase the exposure. On the other hand, the bone fractures cause large deformation, hence preoperative and postoperative structures of the region of interest are significantly different, and one cannot benefit from prior scans for alignment. Lastly, incorporating external trackers leads to an increase in surgical complexity and line of sight problem. In this study, we propose a novel stitching approach, using a co-registered X-ray source with an optical camera attached to the C-arm, and a patient positioning-laser to recover the depth scale. Therefore, the system is mobile, self-contained and independent of the OR, and the workflow remains intact. It could be deployed after a single factory calibration. The alignment transformation of volumes is computed based on the video frames, and prior models are not required. We target cases with large gaps between the volumes and focus the approach on spatial alignment of separated regions of interest. Image quality will remain intact, and the radiation dose will be linearly proportional to the size of the individual non-overlapping sub-volumes of interest.

Materials and Methods

System Setup and Calibration

The CBCT-enabled motorized C-arm is positioned relative to the patient by utilizing the positioning-lasers, which are built into the image intensifier and C-arm base. To enable the stitching of multiple sub-volumes, the transformation of the patient relative to the C-arm center must be recovered. In contrast to existing techniques we do not require additional hardware setup around the C-arm, but we attach a camera to the C-arm in such manner that it does not obstruct the surgeons access to the patient. By using one mirror, the camera and the X-ray source centers are optically identical. The system setup is outlined in FIG. 2.

The system is composed of a mobile C-arm, ARCADIS® Orbic 3D, from Siemens Medical Solutions and an optical video camera, Manta® G-125C, from Allied Vision Technologies. The C-arm and the camera are both connected via ethernet to the computer with custom software to store the CBCT volumes and video. The X-ray and optical images are calibrated in an offline phase.

The positioning-laser in the base of the C-arm spans a plane, which inter-sects with the unknown patient surface, and can be observed as a curve in the camera image. To determine the exact position of the laser relative to the camera, we perform a camera-to-plane calibration. Multiple checkerboard poses (n) are recorded for which the projection of the positioning-laser intersects with the origin of the checkerboard. Once the camera intrinsics are estimated, the camera-centric 3D checkerboard poses are computed. Under the assumption that the 3D homogeneous checkerboard origins,

x⁽³⁾={xᵢ|xᵢ=[x,y,z,1]^{T}}ᵢ₌₀ⁿ

[wherein (2) and (3) denote 2D and 3D points; (s) denotes points up to a scale] lay on the laser plane, the plane coefficients A=[a, b, c, d] are determined by performing RANdom SAmple Consensus (RANSAC) based plane fitting to the observed checkerboard origins, which attempts to satisfy:

$${arg min}_{A} {\sum }_{{x}_{j} ∈ Ω} \vert {Ax}_{j} \vert ,$$

where Ω is subset of checkerboard origins, which are inliers to the plane fitting.

CBCT Volume and Video Acquisition

To acquire a CBCT volume, the patient is positioned under guidance of the lasers. Then, the motorized C-arm orbits 190° around the center visualized by the laser lines, and automatically acquires a total of 100 2D X-ray images. The reconstruction is performed using the Feldkamp method, which utilizes filtered back-projection, resulting in a cubic volume with a 256 voxels along each axis and an isometric resolution of 0.5 mm. During the re-arrangement of C-arm and patient for the next CBCT acquisition, the positioning-laser is projected at the patient, and each video frame is recorded. For simplicity, we will assume that in the following the C-arm is static, while the patient is moving. However, as only the relative movement of patient to C-arm is recorded, there are no limitations on allowed motions.

Two-Dimensional Feature Detection and Matching

The transformation describing the relative patient motion observed between two video frames is estimated by detecting and matching a set of natural surface features and the recovery of their scale. For each frame, we automatically detect Speeded Up Robust Features (SURF) as previously described, which are well suited to track natural shapes and blob-like structures. To match the features in frame k to the features in frame k+1, we find the nearest neighbor by exhaustively comparing the features, and removing weak or ambiguous matches. Outliers are removed by estimating the Fundamental Matrix, Fk, using a least trimmed squares formulation and rejecting up to 50% of the features, resulting in a set of nk features

fₖ⁽²⁾={f_{k,j}|f_{k,j}=[x,y,1]^{T}}ⱼ₌₁ⁿ^{ₖ}

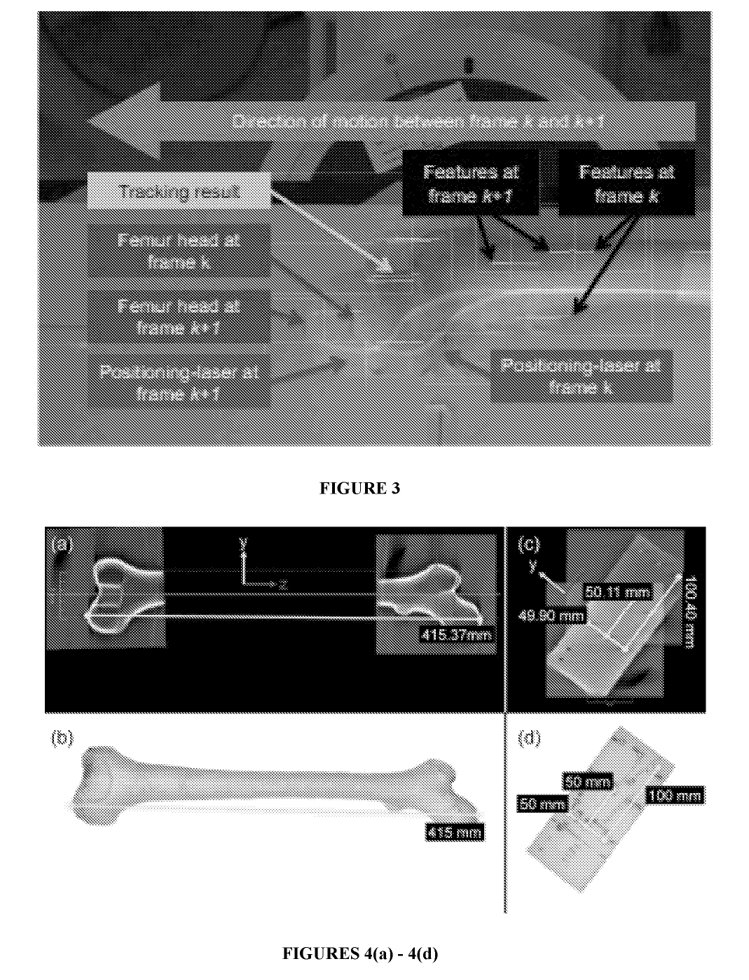

in frame k (see FIG. 3). To estimate the 3D transformation, the 3D coordinates of this set of features need to be estimated.

Recovering Three-Dimensional Coordinates

In each frame k, the laser is automatically detected. First the color channel corresponding to the laser's color is thresholded and noise is removed by analyzing connected components. To find the mk 2D points,

pₖ⁽²⁾={p_{k,i}|p_{k,i}=[x,y,1]^{T}}ᵢ₌₁^{m}^{ₖ}

which are most likely on the plane, the resulting binary image is thinned. Each 2D laser point p(2) is projected back to a point up to a scale

p_{k,i}^{(s)}=[x_{k,i}^{(s)},y_{k,i}^{(s)},1,1]^{T}

using the Moore-Penrose pseudo-inverse of the camera projection matrix, P:

p_{k,i}⁽³⁾=s_{k,i}p_{k,i}^{(s)}=s_{k,i}P⁺p_{k,i}⁽²⁾, (2)

where the scale sk,i is recovered by intersecting the point up to a scale

p_{k,i}^{(s)}

with the plane:

$${s}_{k , i} = \frac{- d}{{ax}_{k , i}^{( s )} + {by}_{k , i}^{( s )} + c} . \qquad ( 3 )$$

Once the 3D laser points are recovered, the scale for each feature,

f_{k,j}^{(s)}=s_{k,j}P⁺f_{k,j}⁽²⁾.

can be estimated by interpolating the scales of the closest points

p_{k,i}⁽³⁾.

Estimating 3D Transformation and CBCT Volume Stitching

After the estimation of the 3D coordinates of the matched features, the transformation for the frames k and k+1 is computed by solving the least squares fitting for two sets of 3D points, obtaining the transformation matrix Tk. Note that, only features in a small neighborhood of the laser line, <1 cm, are used. Hence, features on other body parts, e.g. the opposite leg, are discarded. To verify the estimated transformation, the Iterative Closest Point (ICP) algorithm is used to perform a redundancy test using the laser points. In other words, ICP is applied after transforming the laser points

pᵢ⁽³⁾

from frame k to the next k+1 only for verification. Consequently, for long bones, translation along the laser line is not lost. This results in a transformation

\hat{T}ₖ.

If value is not nearly identity, the frame k+1 is rejected and the frames k and k+2 are used to compute

\hat{T}ₖ.

To obtain the overall transformation TCBCT, all transformations

Tₖ∈Γ are accumulated,

where Γ is the domain of all valid transformations:

$${T}_{CBCT} = {}_{}^{CBCT}{T}{}_{camera}^{} {\prod }_{{T}_{k} ∈ Γ} {T}_{k} , \qquad ( 4 )$$

where ^{CBCT}T_{camera} is the transformation from camera coordinate system to the CBCT coordinate system obtained during calibration.

Experiments and Results

The novel laser-guided stitching method is evaluated in two different, but realistic scenarios. For each phantom, we performed vision-based stitching and evaluated the quality by measuring 3D distances in the stitched volumes and real object. In addition, the stitching quality was compared to intensity-based mosaicing using overlapping CBCT volumes, indicating the accuracy of the overall 3D transformation T_{CBCT}.

The result of vision-based stitching is illustrated in FIG. 4 (a) on the long bone phantom in the absence of overlaps, and in FIG. 4 (c) on the fiducial phantom with overlaps. The absolute distances are compared to real world measurements which are illustrated in FIGS. 4 (b) and (d). Detailed results are reported in table 1, which shows the differences of measurements of the vision-based stitched CBCT volumes and real objects. The errors are apportioned according to the coordinate frames illustrated in FIG. 4, while the norm reflects the overall error. In addition, the absolute distance error reports the percentage of error with respect to the absolute distances measured. Average errors are in the range of 0.65±0.28 mm and 0.15±0.11 mm for long bone and fiducial phantom stitching, respectively. Lastly, for overlapping volumes, we have compared the vision- and intensity-based stitching by performing rigid registration using normalized cross correlation as similarity measure. The intensity-based stitching deviated from the vision-based stitching by 0.23 mm, indicating an overall good alignment.

Discussion and Conclusion

The proposed technique is an overlap-independent, low dose, and accurate stitching method for CBCT sub-volumes with minimal increase of workflow complexity. We attached an optical camera to a mobile C-arm, and used the positioning-laser to recover the 3D depth scales, and consequently aligned the sub-volumes. As a result of this method, the stitching is performed with low dose radiation, linearly proportional to the size of non-overlapping sub-volumes. We expect this to be applicable to intraoperative planning and validation for long bone fracture or joint replacement interventions, where multi-axis alignment and absolute distances are difficult to visualize and measure from the 2D X-ray views.

Our approach does not limit the working space, nor does it require any additional hardware besides a simple camera. The C-arm remains mobile and in-dependent of the OR. One requirement is that the C-arm does not move during the CBCT acquisition, but we believe that the use of external markers could solve this problem and may yield a higher accuracy. However, in our scenario we intentionally did not rely on markers, as they would increase complexity and alter the surgical workflow. Our approach uses frame-to-frame tracking, which can cause drift. In fact, the ICP verification helps us to detect such drifts as it is based on points which were not used for motion estimation. Therefore, if the estimated motion from ICP increases over time, we can detect the drift and use ICP to correct if necessary. Alternatively, the transformations could be refined using bundle adjustments. Further studies on the effectiveness during interventions are underway. Also, the reconstruction of the patient surface during the CBCT acquisition may assist during the tracking of the patient motion.

### Example 2

### Preclinical Usability Study of Multiple Augmented Reality Concepts for K-Wire Placement

Summary

Purpose

In many orthopedic surgeries, there is a demand for correctly placing medical instruments (e.g., K-wire or drill) to perform bone fracture repairs. The main challenge is the mental alignment of X-ray images acquired using a C-arm, the medical instruments, and the patient, which dramatically increases in complexity during pelvic surgeries. Current solutions include the continuous acquisition of many intra-operative X-ray images from various views, which will result in high radiation exposure, long surgical durations, and significant effort and frustration for the surgical staff. This work conducts a preclinical usability study to test and evaluate mixed reality visualization techniques using intra-operative X-ray, optical, and RGBD imaging to augment the surgeon's view to assist accurate placement of tools.

Method

We design and perform a usability study to compare the performance of surgeons and their task load using three different mixed reality systems during K-wire placements. The three systems are interventional X-ray imaging, X-ray augmentation on 2D video, and 3D surface reconstruction augmented by digitally reconstructed radiographs and live tool visualization.

Results

The evaluation criteria include duration, number of X-ray images acquired, placement accuracy, and the surgical task load, which are observed during 21 clinically relevant interventions performed by surgeons on phantoms. Finally, we test for statistically significant improvements and show that the mixed reality visualization leads to a significantly improved efficiency.

Conclusion

The 3D visualization of patient, tool, and DRR shows clear advantages over the conventional X-ray imaging and provides intuitive feedback to place the medical tools correctly and efficiently.

Introduction

A continuous and rapid evolution of technology has changed the face of trauma and orthopedic surgeries in the past decades. Especially, minimally invasive techniques are widely accepted for treatment of bone fractures in spine and pelvis, thanks to the development of modern imaging technology and computer-aided navigation systems. The benefits of minimally invasive orthopedic surgeries are the reduction in blood loss, collateral tissue damage, and overall operating duration. However, these techniques usually yield a higher X-ray exposure for both patient and clinical staff and may increase fatigue and frustration due to the difficulty in continuous repositioning of the mobile X-ray machine (C-arm).

The main challenge during percutaneous K-wire placement and screw fixation is the mental alignment of patient, medical instruments, and the intra-operative X-ray images, which also requires the frequent repositioning of the C-arm. For instance, in pelvic acetabulum fractures, the surgeon needs to find the correct trajectory of the K-wire through a small bony structure, namely the superior pubic ramus. The misplacement of the K-wire could cause severe damage to the external iliac artery and vein, obturator nerve, or structures such as the inguinal canal and intra-articular hip joint. It is not unusual that a single K-wire placement for one screw takes up to ten minutes.

The standard treatment procedure for undisplaced superior pubic ramus fractures requires several K-wire placements and subsequent screw insertions. For each K-wire, the surgeon first locates the entry point location and performs a skin incision at the lateral side of the hip, which requires several intra-operative X-ray images from various perspectives to confirm the exact tool orientation. It is common to correct the K-wire placement. While advancing the K-wire through soft tissue and into the bone, X-ray images from various perspectives are acquired to constantly validate the trajectory. The path is narrow through the superior pubic ramus. After the K-wire is placed, the procedure concludes by drilling and placing a cannulated screw. Computer-aided surgical navigation systems have been introduced to assist the placement of K-wires and screws. Current solutions use preoperative computed tomography (CT) volumes, external optical tracking systems, and tracked markers as reference on medical instruments, the patient, and the C-arm. Navigation systems then provide intra-operative information on the spatial relation of surgical instruments and medical images. The validation of the K-wire placement is performed using conventional X-ray imaging.

The benefits of navigation systems are controversial. Some publications indicate a reduction in the radiation dose and an increase in accuracy, while a more recent study shows no clear advantage of using navigation systems in some procedures. A major drawback of navigation systems is the high cost, which limits the availability of such systems to major hospitals and research facilities. The cost is driven by external hardware, which constitutes a logistical problem due to the bulkiness and consumption of space in the OR. Beyond hardware requirements, the systems also impose a change in the surgical workflow. In summary, after two decades of surgical navigation systems, expert surgeons are starting to realize that these systems have failed to provide the advantages promised. They do not reduce the required OR time, show no systematic, significant influence on the patient outcome, and reduce the frustration of the surgeon and staff. The additional efforts required to use modern surgical navigation systems often outweigh the benefits in many scenarios. Therefore, interventions are frequently performed without surgical navigation systems even though navigation would be available and theoretically present a benefit, which has been especially researched for spine surgery.

An alternative solution, which is comparatively inexpensive, contained in existing equipment and intuitive, has been proposed. This solution adds a mirror and video camera to a C-arm, such that the X-ray and optical views align. After a single calibration and warping step, the video view can be augmented with the X-ray images, which pro-vides an intuitive optical feedback to the surgeon. In cadaver studies, this system leads to reduced radiation dose and increase in surgical efficiency in terms of duration and accuracy. During orthopedic and trauma procedures, the use of a camera augmented intra-operative X-ray system resulted in improved incisions, reduced radiation exposure of the surgeon, and simplified instrument tool alignment. However, the mirror construction reduces the free moving space of the surgeon, which can be overcome in mounting the camera next to the X-ray source. That setup will only be able to augment the video view with warped X-ray images, which are clinically less relevant. Both approaches require the X-ray source to be positioned on the top rather than below the surgical table, which is an unusual setup and may increase the exposure of the surgeon to scatter radiation. A red-green-blue depth (RGBD) camera was mounted to a C-arm instead of a video camera. Similarly to an RGB camera, an RGBD camera provides a 2D color image and additionally provides a depth value for every pixel which represents the distance between the observed object and the camera origin. This allows to reconstruct the 3D surfaces of an object. The system using the RGBD camera, rather than the RGB camera, enables an offline 3D/2D mixed reality visualization of X-ray on the reconstructed patient surface. The main limitation of this work is due to 2D projective nature of the X-ray image. As soon as the display viewpoint of the surface is different than the X-ray source optical axis, the visualization is physically wrong. Using CBCT may allow to overcome this issue, since a new simulated X-ray (DRR) corresponding to the viewpoint can be generated dynamically. Previously, two RGBD sensors were mounted on a mobile C-arm in order to synthesize the video as seen from the X-ray source viewpoint without the need of a mirror construction.

The integration of a stereo camera near the X-ray detector enables tool tracking within the working space of the C-arm. If CT images are transferred to the inter-operative setup, a digitally reconstructed radiograph (DRR) can be computed and augmented onto the one camera view. This system has been presented as a good combination of augmented reality visualization and surgical navigation systems, but requires markers on the patient and tools. The change in the augmented view requires the movement of the entire system and may introduce errors of the alignment of CT, and optical view in case the patient marker is occluded.

Systems with augmented video may benefit of the use of RGBD cameras, which allows the positioning of the virtual cameras and renderings of the patient surface from arbitrary perspectives. RGBD information can also be used to improve the understanding of the environment and enhance the augmentation.

In this example, a preclinical usability study is set forth to provide a more comprehensive understanding whether enhanced C-arm systems provide a clinically relevant bene-fit. We will compare K-wire placement using (i) conventional X-ray imaging, (ii) 2D RGB video augmented with X-ray images, and (iii) a novel 3D RGBD video augmented with DRRs generated from cone beam CT (CBCT). The later sys-tem allows the surgeon virtually rotate the entire scene (DRR, patient surface, and tools) and simultaneously view the scene from different perspectives. A total of 21 K-wire placements are performed by seven surgeons, ranging from residents to attending physicians. We compare the system usabilities in terms of surgical efficiency, which is defined by the number of X-ray images, duration, accuracy, and surgical task load.

Method

In this section we first describe the imaging systems to be compared. These include conventional intra-operative X-ray imaging, X-ray image augmented 2D video, and a novel 3D RGBD view augmented with DRR. Finally, we present the questionnaires and statistical methods to perform the usability study.

Imaging Systems

To evaluate the usability of mixed reality visualization techniques, we acquire a baseline using conventional intra-operative X-ray imaging.

Conventional Intra-Operative X-Ray Imaging

This imaging method using a standard C-arm provides the baseline performance as it is the most commonly used system to perform image-guided K-wire placement. The images are obtained in the digital radiography (DR) mode. This allows for a single, brief exposure at higher than normal mA to capture a higher-quality single image. For reasons of comparability between subjects, we limit the functionality of the C-arm to this mode. 2D RGB video and X-ray visualization To achieve a fused RGB and X-ray visualization, we attached a camera near the X-ray source. Using a mirror construction, the X-ray source and optical camera centers are virtually aligned as previously described. To be able to observe the surgical site using the RGB camera, the X-ray source and camera are positioned above the patient.

The X-ray images are obtained using the standard C-arm in DR mode. After camera calibration, the alignment registration of optical and X-ray images is performed using a single plane phantom with radiopaque markers that are also visible in the optical view.

Finally, this first augmented reality system allows the simultaneous display of live RGB video overlaid with DR images obtained at the user's discretion. Additionally, we provide the user with the option to control the alpha blending to change the transparency to be able to focus on the X-ray image or video background.

3D RGBD and DRR Via CBCT Visualization

The previous system requires the repositioning of the C-arm in order to change the optical and X-ray view. To overcome this limitation, we introduce a novel system using an RGBD camera and cone beam CT (CBCT) volumes, which allows the simultaneous visualization of the patient and medical data from multiple arbitrary views. As the RGBD camera is rigidly mounted to the X-ray detector, the X-ray source can be positioned under the surgical table as done during conventional image-guided surgery.

To calibrate the RGBD information and the CBCT volume, we simultaneously acquire the CBCT and the surface information using the RGBD camera of an arbitrary object. We extract the surface from the CBCT by simple thresholding, and reconstruct the surface observed by the RGBD camera as previously described, resulting in a smooth and precise surface mesh. The calibration is obtained by means of surface matching.

After the calibration is obtained, the CBCT and patient's surface scan are acquired. These data are fused into a mixed reality scene, in which the patient's surface, DRR from CBCT, and live RGBD data (e.g., hand or tool) are visualized. The surgeon can now define multiple arbitrary views of the fused DRR and RGBD data. The system allows perspectives that are usually not possible using conventional X-ray imaging, as the free moving space is limited by the patient, surgical table, or OR setup. The live RGBD data provide an intuitive understanding of the relation of CBCT volume, patient's surface, surgeon's hand, and medical tools.

Evaluation Method

During the usability study, we evaluate the performance achieved using each system. Our hypothesis states that the mixed reality visualizations improve the surgical efficiency. Our data cannot be assumed to be of normal distribution, but are ordinal. Using Friedman's ANOVA, we test whether the differences in observations are coincidental or statistically significant. Additionally, we need to test whether the individual systems yield a significant difference in terms of the surgical efficiency. As a normal distribution of our data cannot be assumed, these post hoc tests are performed using the Wilcoxon signed-rank tests with Bonferroni correction.

Surgical Efficiency Measure

Together with our clinical partners, we identified following measures to express the surgical efficiency. First, the duration of each K-wire placement is of importance. During hip surgeries, this process is often the most time-consuming and is followed by a relatively quick drilling step and screw placement. Surgical navigation systems often do not yield the advantage of reducing the overall OR time. Next, the number or X-ray images and cumulative area dose product is of importance to the both patient and surgeon. During conventional C-arm guided placement, a large number of X-ray images are acquired during the planning and propagation of the K-wire. One of our systems will acquire a preincision CBCT, for which we will include the dose into our statistics. Finally, the error is defined by the medical need of the K-wire remaining in the superior pubic ramus. We will compute the average distance between the ideal path, which is the center line of bone phantom, and the placement of it. However, as all study participants are trained surgeons, we do not expect that any significant improvement will be possible.

Surgical Task Load

The workload is measured using a standardized questionnaire, namely the Surgical Task Load Index (SURG-TLX). This test is designed to evaluate the mental demands, physical demands, temporal demands, task complexity, situational stress, and distractions during surgical interventions. It is specifically designed and validated to analyze implications for categorizing the difficulty of certain procedures and the implementation of new technology in the operating room.

Experiments

The K-wire placement through the superior pubic ramus (acetabulum arc) is a complex and cumbersome procedure, which is performed frequently and in case of an undislocated fracture usually minimally invasive. In our experiments, we mimicked this scenario by designing adequate radiopaque phantoms. The surgeons each performed three K-wire placements using the image-guidance systems in a randomized order.

Phantom Design

The superior pubic ramus is a thin tubular bone with an diameter around 10 mm. In case of an undislocated fracture, a 2.8-mm-thin K-wire needs to be placed through a narrow safe zone. Later, a 7.3 mm cannulated screw is inserted. Our phantom was created out of methylene bisphenyl diisocyanate (MDI) foam, which is stiff, lightweight, and not radiopaque. The bone phantom was created out of an thin aluminum mesh filled with MDI. The begin and end of the bone were marked with a rubber radiopaque ring. Therefore, the bone phantom is very similar to the superior pubic ramus in terms of haptic feedback during K-wire placement, as the K-wire will easily exit the bone without significant resistance. The orientation of the bone within the phantom was randomized and phantoms were not reused for other experiments.

Experimental Setup and Design

In all our experiments, we use a CBCT enabled C-arm, SIEMENS ARCADIS® Orbic 3D from Siemens Healthcare GmbH, which automatically computes the cumulative area dose for the patient for each imaging session. The second and third system use an optical video camera, Manta G-125C®, from Allied Vision Technologies, or an RGBD camera, Intel Real Sense® Camera (F200), Intel Corporation, respectively.

Each surgeon was asked to perform three independent K-wire placements using the different imaging modalities. The order of the modalities was randomized, but for simplicity we will refer to the first (S1), second (S2), and third system (S3) in the order presented in “Imaging systems” section. Using a 2.8 mm K-wire, the surgeons identified the entry point on the phantom's surface, drilled toward the begin of the bone phantom, and passed through the tubular bone structure. When finished, the K-wire was removed from the drill and a CBCT was acquired to measure the error of the K-wire placement postoperatively.

Results

We observed a total of 21 minimally invasive K-wire placements using different image-guidance systems. Table 2 shows the observed time in seconds, number of acquired X-ray images, cumulative area dose product (dose) in cGycm2, error relative to the ideal path in mm, and surgical task load index for each participant and system used. Note that the task load is a accumulative scale, for which the score of 5 and 100 represents the lowest and highest possible load, respectively. The aggregated observations are presented in Table 3.

TABLE 2
This table presents all observed values for each study participant and system used.

| | Participants | | | | | | |
|---|---|---|---|---|---|---|---|
| | 1 | 2 | 3 | 4 | 5 | 6 | 7 |
| System 1: | | | | | | | |
| Conventional C-arm | | | | | | | |
| Time (sec) | 937 | 686 | 617 | 464 | 636 | 388 | 432 |
| X-ray images | 80 | 47 | 44 | 33 | 32 | 21 | 29 |
| Dose (cGycm²) | 7.68 | 1.73 | 3.54 | 4.38 | 5.62 | 2.69 | 5.38 |
| Error (mm) | 3.08 | 7.88 | 11.43 | 3.01 | 1.87 | 2.27 | 2.72 |
| Task load | 76 | 25.67 | 41.67 | 17.67 | 53.33 | 19.33 | 70.67 |
| System 2: RGB and | | | | | | | |
| X-ray visualization | | | | | | | |
| Time (sec) | 360 | 431 | 521 | 295 | 436 | 691 | 768 |
| X-ray images | 19 | 13 | 20 | 13 | 18 | 20 | 30 |
| Dose (cGycm²) | 3.07 | 1.3 | 1.57 | 1.92 | 1.42 | 2.38 | 5.56 |
| Error (mm) | 7.92 | 2.69 | 3.85 | 4.23 | 4.88 | 3.44 | 1.74 |
| Task load | 60.33 | 10 | 20 | 21.67 | 26 | 22.33 | 62.33 |
| System 3: RGBD and | | | | | | | |
| DRR visualization | | | | | | | |
| Time (sec) | 182 | 180 | 380 | 181 | 190 | 254 | 339 |
| X-ray images | 1 | 2 | 2 | 2 | 2 | 3 | 3 |
| Dose (cGycm²) | 1.76 | 1.9 | 1.48 | 1.44 | 1.55 | 1.47 | 1.59 |
| Error (mm) | 7.38 | 6.39 | 8.45 | 6.53 | 1.39 | 2.31 | 3.48 |
| Task load | 20.33 | 5 | 24.33 | 23 | 11.33 | 8.67 | 30.33 |

For the RGBD and DRR visualization, a CBCT was acquired, which is included in the dose measurement, but not in the number of X-ray images acquired.

TABLE 3
Accumulated values and standard deviations for the observations
(Table 2) are presented in this table.

| | S1: C-arm | | S2: RGB/X-ray | | S3: RGBD/DRR | |
|---|---|---|---|---|---|---|
| Time (sec) | 594 ± | 188 | 500 ± | 172 | 243 ± | 84 |
| X-ray images | 40.86 ± | 19.38 | 19.00 ± | 5.72 | 2.14 ± | 0.69 |
| Dose (cGycm²) | 4.43 ± | 2.00 | 2.46 ± | 1.50 | 1.60 ± | 0.17 |
| Error (mm) | 4.61 ± | 3.62 | 4.11 ± | 1.97 | 5.13 ± | 2.72 |
| Task load | 43.48 ± | 24.03 | 31.81 ± | 20.76 | 17.57 ± | 9.33 |

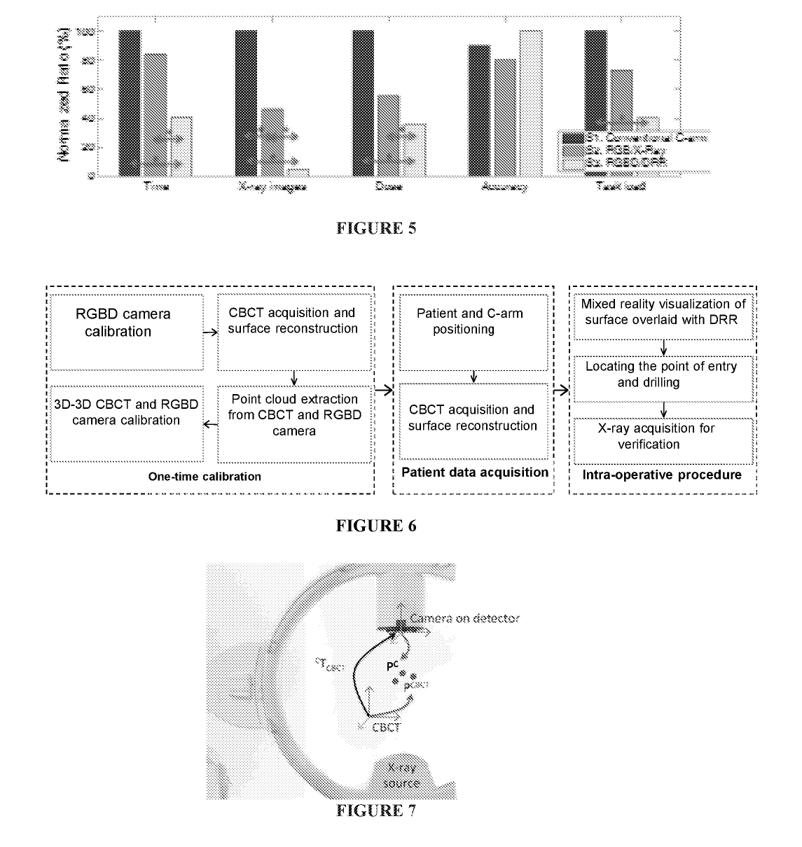

When comparing the use of a conventional C-arm to the use of a mixed reality system, a clear tendency toward a decreased operation time, lower number of X-ray images acquired, reduced dose, and reduced task load can be observed, as shown in FIG. 5, is observed. The measure of the dose includes the acquisition of the CBCT volume required for the RGBD and DRR visualization (system 3). The accuracy does not improve, as it is already in an acceptable range.

Statistical Evaluation

Statistical tests were performed to study the changes in the surgical efficiency measures. Significance is achieved for p-values lower than 0.05, indicating that the chance of the change being coincidentally observed is less than 5%. A Friedman test was calculated to compare each measure as a normal distribution of the data could not be assumed. We found a significant difference in time (χ2(3)=11.14, p<0.01), number of X-ray images (χ2(3)=12.29, p<0.01), and radiation dose (χ2(3)=6.00, p<0.05) depending on the kind of assistance that was provided to the subjects. The post hoc tests were computed using the Wilcoxon signed-rank tests with Bonferroni correction.

Time

The tests show significant differences between the first system (S1: conventional C-arm) and the third system (S3: RGBD and DRR Visualization) (Z=−2.366, p<0.05), and significant differences between second system (S2: RGB and X-ray visualization) and S3 (Z=−2.366, p<0.05). This indicates that the 3D placement of the K-wire is best supported with a multi-view 3D visualization.

X-Ray Images

All combinations of S1, S2, and S3 show a significant reduction in the number of X-ray images acquired: S1 to S2 (Z=−2.117, p<0.05), S2 to S3 (Z=−2.375, p<0.05), and S1 to S3 (Z=−2.366, p<0.05).

Radiation Dose

Although we have included the dose caused by the CBCT in S3, the tests show that the intervention using the conventional C-arm causes a significantly higher cumulative area dose product: S1 to S2 (Z=−2.197, p<0.05), S1 to S3 (Z=−2.197, p<0.05). However, the dose difference between S2 and S3 is not significant.

Error

No significant difference in error between based on the use of different systems can be observed. Therefore, the reported changes in accuracies are most likely coincidental.

Surgical Task Load Index

Similarly to the changes in the duration of the intervention, the reduction in task load evaluated using the SURG-TLX® is only significant between S1 and S3 (Z=−2.197, p<0.05).

In conclusion, S3 yields better results in terms of all observed surgical efficiency measures except for the accuracy, for which the difference is not statistically significant. Even though S3 is not a fully developed product, our usability study indicates that there are clear advantages over the conventional C-arm system when guiding K-wire placement.

Discussion and Conclusion

In this example, we presented a usability study using three different mixed reality visualization systems to perform K-wire placement into the superior pubic ramus. This procedure was chosen because of the high clinical relevance, frequent prevalence, and the especially challenging minimal invasive surgical technique.

Our attention was focused on the usability and clinical impact of the three different visualization systems. For that reason, we were interested not only in the quality of a procedure (e.g., accuracy), but also in the workload and frustration that the surgeons experienced while using the different systems. We observed the 21 interventions performed by seven surgeons and used the Surgical TLX® to evaluate the task load.

Our results show that the 3D visualization yields the most benefit in terms of surgical duration, number of X-ray images taken, overall radiation dose, and surgical workload. This is despite the fact that the mixed reality visualizations currently do not provide an augmentation of a tracked tool. The conventional C-arm constitute the system yielding the poorest results, indicating a high potential for improvements to the currently used image-guidance systems. In all scenarios, the surgeons placed the K-wire within clinically relevant tolerance. The change in accuracy of the placed K-wire is not significant, which shows that all three systems provide sufficient support in terms of placement quality.

This study also showed the clear necessity to continue research and development of the mixed reality systems. For instance, movement of the C-arm or surgical table may lead to loss of tracking, which results in an outdated mixed reality visualization. However, in a clinical scenario, the failure of the mixed reality system is immediately visible and the surgeon can continue using the conventional X-ray imaging capabilities.

In our evaluation, we have not taken the learning curve under consideration as we frequently observed that surgeons unfamiliar to the mixed reality system adopted very quickly. Perhaps an initial training phase would further emphasize the advantages of the augmentations.

Future studies will include other complex K-wire placement procedures, such as performed in case of an iliosacral fracture, or pedicle screw placement. We will attempt to include even more surgeons during the next studies, which will allow for a more detailed statistically analysis. Addition-ally, we will investigate the usability for other procedures, such as Jamshidi needle placement or general needle biopsies.

Our usability study showed that mixed reality systems have great potential to increase surgical efficiency, and should be in the focus of research on computer-assisted interventions. The integration of better visualization techniques or tool tracking and identification may yield more opportunities in assisting surgeons to conduct interventions more efficiently and reduce the task load.

### Example 3

### Calibration of RGBD Camera and Cone-Beam CT for 3D Intra-Operative Mixed Reality Visualization

Summary

Purpose

This work proposes a novel algorithm to register cone-beam computed tomography (CBCT) volumes and 3D optical (RGBD) camera views. The co-registered real-time RGBD camera and CBCT imaging enable a novel augmented reality solution for orthopedic surgeries, which allows arbitrary views using digitally reconstructed radiographs overlaid on the reconstructed patient's surface without the need to move the C-arm.

Methods

An RGBD camera is rigidly mounted on the C-arm near the detector. We introduce a calibration method based on the simultaneous reconstruction of the surface and the CBCT scan of an object. The transformation between the two coordinate spaces is recovered using Fast Point Feature Histogram descriptors and the Iterative Closest Point algorithm.

Results

Several experiments are performed to assess the repeatability and the accuracy of this method. Target registration error is measured on multiple visual and radio-opaque landmarks to evaluate the accuracy of the registration. Mixed reality visualizations from arbitrary angles are also presented for simulated orthopedic surgeries.

Conclusion

This is the first calibration method which uses only tomographic and RGBD reconstructions. This means that the method does not impose a particular shape of the phantom. We demonstrate a marker-less calibration of CBCT volumes and 3D depth cam-eras, achieving reasonable registration accuracy. This design requires a one-time factory calibration, is self-contained, and could be integrated into existing mobile C-arms to provide real-time augmented reality views from arbitrary angles.

Introduction

X-ray imaging is an important tool for percutaneous iliosacral and pedicle screw placements in spine surgeries. To avoid potential damages to soft tissues and the nervous system near the vertebra, and reduce muscle retraction, significant amount of fluoroscopic/X-ray images are acquired from multiple views during these interventions. Foreign body removal surgeries also require a high number of X-ray image acquisitions, as there are significant risks of inadequately performing the wound debridement. Multiple attempts to remove them could lead to larger incisions, additional trauma, delay in healing, and worsened outcomes.

To place or remove a rigid object during minimally invasive image-guided orthopedic operations, the surgeon first locates the point of entry on the skin by acquiring multiple X-ray images from different views while having a tool for reference in the scene. The reference tool (for instance a guide wire with 2.8 mm diameter) is used during the intervention to assist the surgeons with the mental alignment.

An exemplary workflow involves the collection of a set of anteroposterior X-ray images in which the target anatomy and the guide wire are visible. Next, the direction of the medical instrument is corrected in corresponding lateral and oblique views, which may introduce small displacements in the anteroposterior side. To ensure the accurate placement of the medical instrument, this procedure is repeated several times, and during each iteration the guide wire is traversed further through the tissue until the target is reached. Most commonly, the bone structure is between 5 mm (vertebra) and 12 mm (superior pubic ramus) in diameter, and the diameter of the screw is between 2 and 7.3 mm depending on the application. Lastly, images are acquired to validate that the screw remains within the bone, and depending on the performed placement the procedure may need to be repeated.

Delicate operations of these types have long surgical durations as they require the acquisition of many intra-operative images which implies frequent C-arm repositionings. This leads to a high surgical effort and major frustration for the surgical staff, long procedures, and high radiation exposure. This is in contrast to the need of shortening anesthesia durations for elderly patients due to postoperative complications. Thus, it is a challenge for the surgeon to operate accurately in a limited time and minimizing collateral damage to sur-rounding tissue.

External surgical navigation systems are used to provide the spatial relation among the anatomy in medical images, the patient's body in the operation room (OR), and the surgical tool. This information is used to avoid potential damage to surrounding tissue. Alternatively, additional sensors such as cameras may directly be attached to the C-arm to enable tracking of the surgical tools. The data acquired from these sensors could be used together with medical data to provide intuitive visualizations.

Optical-based image-guided navigation systems were used to recover the spatial transformation between surgical tools and a 3D rotational X-ray enabled C-arm with submillimeter accuracy. Significant reduction in radiation exposure was achieved by navigating the surgical tool together with a tracked C-arm with markers attached to the detector plane. Navigation-assisted fluoroscopy in minimally invasive spine surgery with optical tracker for placing pedicle screws was evaluated in. Both publications reported a reduction in radiation exposure. However, no statistically significant changes in the time of surgery was found. There are two main problems associated with these systems: First, they increase the complexity of the surgery, require additional hardware, occupy significant amount of space, and require line of sight between patient and hardware. Second, the surgeon performs the surgery by only observing a display, which does not provide visual feedback of actual patient and current deformation of tissue. Good outcomes highly depend on the surgeon's experience, confidence, and surgical skills. Alternative systems are fully integrated within the absolutely necessary C-arm and range from systems with video cameras to solutions with integrated tracking devices. A video camera attached to a mobile C-arm was co-registered with an X-ray source to pro-vide real-time augmentation of the surgical site. X-ray and optical centers are virtually aligned using an X-ray transparent mirror construction and calibrated by utilizing X-ray and optically visible markers. Next, a homography is estimated to warp the optical images to enable the augmentation with the undistorted X-ray image. This system was used in over 40 orthopedics and trauma procedures, and the results indicated improvements in terms of optimal incision, avoidance of direct radiation exposure, and instrument axis alignment. The evaluation through assisted interlocking procedures showed that as long as depth information is not required, the system can assist the surgeon in accurately placing the tools inside the patient.

Similarly, previous studies incorporated a video camera and per-formed an online calibration by placing a metal rod with known shape close to patient's bone. Cadaver studies for screw placement and femoral fractures on real patients reported 26-30% decrease in radiation exposure, but no statistically significant change in the procedure time was reported. In contrast to previous studies, a color and depth camera were mounted on the mobile C-arm to replace the video camera. The 3D/2D mixed reality visualization is demonstrated using a X-ray image augmented on a patient surface offline. The main limitation of this work is due to 2D projective nature of the X-ray image, resulting in a physically wrong visualization as soon as the viewpoint is different from the X-ray source. In one instance, the optical view from the viewpoint of the X-ray source has been synthesized using two RGBD cameras, enabling a system without mirror construction.

A vision-based tracking system using natural features observed in the view of an optical camera attached to a mobile C-arm was suggested to enable the extension of the field of view of CBCT volumes with minimum radiation exposure. Frame-to-frame registration results acquired from the optical camera were applied to CBCT sub-volumes by calibrating CBCT volumes with the optical camera in advance.

An intuitive visualization based on Digitally Reconstructed Radiographs (DRR) was previously proposed and addresses the limitations of conventional navigation systems by providing an augmented reality view of DRR, video and tracked tools. A vision-based navigation is performed by mounting stereo cameras on the C-arm near the detector. Patient and C-arm are then registered using a mixed set of visual and radio-opaque markers on a single calibration phantom. This system reached submillimeter tracking accuracy and has the benefit of not using any external tracking system, thus remaining self-contained. The main complexity arises when visualization from different angles during augmentation requires moving and rotating the C-arm. To put this into perspective, even though the amount of X-ray exposure is decreased and the line of sight issue is minimized, the additional work load of repositioning the C-arm is not eliminated. The main problem associated with RGB cameras is the unknown depth in arbitrary scenes. RGBD cameras are sensing systems capable of acquiring RGB images and co-registered depth information, thus providing the means to a 3D visualization or marker-less tracking. A calibration of an RGBD camera to 2D X-ray images of C-arm was previously proposed. Registration is performed by computing the projection matrix between a 3D point cloud and corresponding 2D points on the X-ray image plane using a visual and radio-opaque planar phantom. This method reached an error for the 2D/3D calibration of 0.54 mm (RMS)±1.40 mm, which is claimed to be promising for surgical applications.

This work introduces a calibration technique for CBCT volumes and RGBD camera and enables an intuitive 3D visualization which overlays both physical and anatomical information from arbitrary views. In contrast to an aforementioned technique, where a 2D video is augmented with 2D DRRs, this technique takes the next step by proposing a full 3D-3D registration and enables the augmentation of a 3D optical view and simulated X-ray images from any arbitrary view. This system is capable of providing views which may be impossible to capture due to a limited free moving space of the C-arm, for instance, intra-operative transversal images. The proposed marker-less vision-based technique only requires a one-time factory calibration as the depth camera and the X-ray source are rigidly mounted together. The calibration repeatability, influence of the point cloud density, and choice of the arbitrary phantom are evaluated in terms of target registration error (TRE).

Method

The proposed technique uses an RGBD camera mounted on a mobile C-arm and recovers a 3D rigid-body transformation from the RGBD surface point clouds to CBCT. The transformation is recovered using Iterative Closest Point (ICP) with a Fast Point Feature Histogram (FPFH) for initialization. The general workflow is illustrated in FIG. 6 and is comprised of an offline calibration, patient data acquisition and processing, and intra-operative 3D augmented reality visualization. The following sections describe the system setup (“System setup” section), calibration phantom characteristics (“Calibration phantom design, point cloud extraction and pre-processing” section), transformation estimation (“Calibration of C-arm and the RGBD camera” section), and the augmented reality overlay (“Mixed reality visualization of DRRs overlaid on the patient's surface” section).

System Setup

The system comprises a mobile C-arm, the SIEMENS ARCADIS® Orbic 3D from Siemens Healthcare GmbH, and a close-range structured-light Intel RealSense® F200 RGBD camera from Intel Corporation which better minimizes the light-power interference and ensures accuracy in shorter ranges compared to time-of-flight or stereo cameras. A structured-light RGBD camera provides reliable depth information by projecting patterned infrared lights onto the surface and computes the depth information based on the pattern deformations. A typical time-of-flight camera, such as the Microsoft Kinect® One (v2), requires additional warm up time of up to 20 min and depth distortion correction. In addition, the depth values highly depend on the color and shininess of the scene objects. On the other hand, conventional stereo cameras require textured surfaces for reliable triangulation, which are not suitable in this application.

The C-arm is connected via Ethernet to the computer for CBCT data transfer, and the RGBD camera is connected via powered USB 3.0 for real-time frame capturing. The RGBD camera is mounted rigidly near the detector, and its spatial position remains fixed with respect to CBCT's origin. After a one-time calibration, the patient is positioned on the surgical table under the C-arm guidance using the laser aiming guide attached to the C-arm. Thereafter, CBCT is acquired, and the surface is scanned using the RGBD camera simultaneously. The system setup is outlined in FIG. 7.

Calibration Phantom Design, Point Cloud Extraction and Preprocessing

A planar checkerboard pattern is used to recover intrinsic parameters of the RGB and depth camera, and their spatial relation. Depth camera intrinsics are used to reconstruct the surface in depth camera coordinates, and the intrinsics of the RGB camera together with their spatial transformation are used for reprojecting the color information onto the surface. For simplicity, we will refer to the calibrated RGB and depth camera as the RGBD camera.

A calibration phantom is introduced into the common view of the CBCT and the RGBD camera. Surface point clouds are then computed from both imaging modalities and are used to estimate a 3D-3D rigid-body transformation. The phantom is composed of three pipes and a cylindrical foam base. Each pipe has a different length and is positioned at diverse height and orientation to pro-vide a unique rigid 3D-3D mapping between two coordinate spaces. Furthermore, the pipes have higher radiation absorption than the foam base, which allows a simple thresholding for point cloud segmentation. In contrast to shape angles or corners, the round surface of the phantom provides a more stable depth information with lower corner reflection effect.

After positioning the calibration phantom at the center of the C-arm, CBCT data are acquired. While the C-arm is rotating, Kinect Fusion® is used to compute the surface reconstruction in RGBD camera space. Raw point clouds Pcr are subjected to least square cylinder fitting for the tubes α, β, and ♥ with known radius r={r_{α}, r_{β}, r_{γ}} and height h={h_{α}, h_{β}, h_{γ}}. Cylinder fitting is performed by minimizing the model function F(⋅) using M-estimator SAmple and Consensus (MSAC):

$$E ( c , u ) = {min}_{c , u ∈ ℝ} {\sum }_{i ∈ {S}_{j}} {F ( {p}_{i}^{C \_ raw} , c , u )}^{2} \\ \vert u \cdot ( {p}_{i}^{C \_ raw} - c ) \vert \leq h / 2 \qquad ( 1 )$$

where c∈R³ is the center, u∈R³ is the orientation of the principle axis of the cylinder, Sⱼ is the sampling set at the jth MSAC iterations, and

F(pᵢ^{cr},c,u) is the residual defined as below where I is the identity matrix:

F(p₂^{cr},c,u)²=pᵢ^{cr}−c)^{I}(I−uu^{I})(pᵢ^{cr}−c)−r² (2)

Next, the pre-processed surface points are computed as the inliers to the parametric model E(c, u) with respect to a distance threshold d:

p^{cr}={p^{cr}(μ);|p^{cr}(μ)−E(c,u)|<d} (3)

Filtering the CBCT data is performed in four steps. First, due to different absorption coefficients of the foam base and the pipes, the intensities are thresholded manually in CBCT data to filter out the foam (image not shown). The remaining points are transformed into mesh grids using fast greedy triangulation algorithm, and an ambient occlusion value is assigned to each vertex. This score defines the amount which each point in the scene is exposed to the ambient light. Higher values are assigned to outer surfaces, and lower values are assigned to interior of the tubes. Lastly, the outer surface is segmented by thresholding the scores of the vertices. The two point clouds P^{C} and P^{CBCT} are used in “Calibration of C-arm and the RGBD camera” section for calibration of CBCT and RGBD data.

Calibration of C-Arm and the RGBD Camera

The RGBD camera is mounted rigidly on the detector of the C-arm as shown in FIG. 7; therefore, the transformation between them remains fixed and could be modeled as a rigid transformation cTCBCT.

To register Pc and PCBCT, ICP is used with an initial guess acquired from a SAmple Consensus Initial Alignment (SAC-IA) with FPFH. FPFH provides a fast and reliable initialization for the two point clouds. To compute the feature histograms (implemented in the Point Cloud Library), the normal

nᵢ^{S}

is estimated for each point in the CBCT and camera space P^{S}, where S∈{C BCT, c}. Next, for each point, a neighborhood Ωi is defined with respect to a given radius. For every point pair

{pⱼ^{S},pₖ^{S}}

inside Ωi, a point feature histogram (PFH) is computed following:

PFHᵢ^{S}=H({ϕⱼₖ^{S}}) (4)

where φ^{S} is the set of angular variations and H(⋅) is the histogram of these features in Ωi. FPFH is then computed as a weighted. PFH in Ωi, where wⱼ is the Euclidean distance between point pair

$$\{ {p}_{j}^{S} , {p}_{k}^{S} \} \text{:}$$

$${FPFH}_{i}^{S} = {PFH}_{i}^{S} + \frac{1}{k} {\sum }_{j ∈ {Ω}_{i}} \frac{1}{{w}_{j}} {PFH}_{j}^{S} \qquad ( 5 )$$

The acquired feature histograms

FPFH^{S}={FPFHᵢ^{S}}ᵢ₌₁ⁿ^{^{S}}

are used in SAC-IA to register the two point clouds iteratively. The transformation T^{(io)} acquired from registration is used as an automatic initialization, and ICP is used to further refine the registration by minimizing the following cost function:

$${}_{}^{C}{T}{}_{CBCT}^{} = {min}_{T} {\sum }_{i , j} {‖ {p}_{i}^{C} - {Tp}_{j}^{CBCT} ‖}_{2}^{2} \qquad ( 6 )$$

Mixed Reality Visualization of DRRs Overlaid on the Patient's Surface

Using the calibration results from “Calibration of C-aria and the RGBD camera” section, an augmented reality over-lay comprised of DRRs rendered from CBCT volumes and the patient's surface is provided. Moreover, by changing the virtual camera pose after the augmentation, the system pro-vides arbitrary perspectives. Finally, background subtraction in the RGBD view enables a real-time visualization of moving point clouds (surgeon's hands and tools). The output is a mixed reality visualization of anatomical data, patient's surface, and surgeon's hands and tools that are useful for orthopedic interventions.

Experimental Validation and Results

Repeatability of Calibration

The repeatability is first assessed by repeatedly performing the calibration using phantoms. For each test, the calibration phantom is placed differently such that all parts are visible in the RGBD view to ensure a full surface reconstruction. The surface reconstruction using an Intel RealSense® RGBD camera on the detector is compared to the reconstruction using a Microsoft Kinect® 360 (v1) camera mounted on the gantry (due to the depth range limitation, the Kinect camera needs to be placed at least 50 cm away from the object). The standard deviation (SD) along x-, y-, z-axis, and rotation Euler angles α, β, γ are shown in Table 4. Results show no significant difference in the variations of the SD (p=0.6547 using Friedmann's test), indicating that the process is repeatable and the calibration algorithm is not camera specific.

TABLE 4
The results of the repeated calibration (five tests) in terms of SD of Euler angles α, β, γ
of ^{c}R_{CBCT}, the x, y, z components of ^{c}t_{CBCT} and || t_{CBCT}||₂, where ^{c}R_{CBCT} and ^{c}t_{CBCT}
are the rotational and translational components of the estimated transformation ^{c}t_{CBCT}.

| | α | β | γ | x | y | z | |
|---|---|---|---|---|---|---|---|
| SD | (rad) | (rad) | (rad) | (mm) | (mm) | (mm) | \|\|^{c}t_{CBCT}\|\|₂ |
| F200 | 0.0045 | 0.0059 | 0.016 | 0.89 | 0.29 | 0.81 | 0.84 |
| Kinect | 0.0047 | 0.0027 | 0.0079 | 0.31 | 0.74 | 0.35 | 0.46 |

Impact of Point Cloud Density

Point clouds acquired from the RGBD camera and CBCT are subjected to downsampling using voxel grid filter with different grid sizes. Results show little affect on the TRE as shown in Table 5. The ICP estimation shows small variations in transformation parameters using the downsampled data. Once the point cloud density of both datasets is below 2.5 mm (less than 3000 points), the initialization using FPFH and the calibration fails.

TABLE 5
The data acquired from the RGBD camera and CBCT contain 25226 and 94547 points, respectively.

| Grid size | No of pts | No of pts | | | | |
|---|---|---|---|---|---|---|
| (mm) | (CBCT) | (depth) | δx | δy | δz | \|\|δ\|\|₂ |
| — | 94,547 | 25,226 | 0.83 ± 0.57 | 0.72 ± 0.52 | 1.40 ± 1.00 | 1.92 ± 0.98 |
| 0.5 | 51,831 | 21,014 | 0.95 ± 0.46 | 0.74 ± 0.63 | 1.58 ± 1.25 | 2.18 ± 1.12 |
| 1.0 | 18,684 | 16,221 | 0.90 ± 0.59 | 0.82 ± 0.50 | 1.52 ± 1.32 | 2.19 ± 1.08 |
| 1.5 | 9016 | 8536 | 0.88 ± 0.52 | 0.95 ± 0.50 | 1.35 ± 0.83 | 2.05 ± 0.62 |
| 2.0 | 5238 | 5183 | 0.89 ± 0.79 | 0.70 ± 0.56 | 1.37 ± 1.06 | 1.96 ± 1.13 |

The TRE for calibrations with downsampled point clouds are shown.
Values are reported as mean ± SD

Impact of Noise

Bilateral filtering is used to remove noise during surface reconstruction. FPFH and ICP are both tolerant to outliers, and thus, little noise does not affect the transformation estimation. Due to the significant difference of the attenuation coefficient of the calibration phantom and the background noise, thresholding the CBCT data eliminates the background noise. Therefore, the calibration algorithm is robust to small amounts of noise and outliers.

Accuracy in Terms of Target Registration Error

To evaluate the accuracy of the calibration, the TRE is computed using a phantom. The phantom contains visual and radio-opaque landmarks and each land-mark is selected manually. TRE is computed as the Euclidean distance between a visual landmark after applying the trans-formation and the corresponding radio-opaque landmark. Since the landmarks are not co-linear nor coplanar, small orientational errors are also reflected in TRE. The accuracy test is repeated three times using eight landmarks. The resulting errors are shown in Table 6. The main misalignment arises from the error in the direction perpendicular to the RGBD camera (due to poor depth quality). In each test, the phantom is placed at a different pose by 90° rotation. Different accuracies among the three tests are mainly due to changes of the overall distance of the landmarks to the camera resulting from their non-uniform distribution.

TABLE 6
The results of the repeated accuracy tests are shown as TRE, where δx,
δy, δz, and ||δ||₂ are the Euclidean distances.

| | δx | δy | δz | \|\|δ\|\|₂ |
|---|---|---|---|---|
| TRE 1 | | | | |
| F200 | 1.26 ± 0.73 | 1.94 ± 1.50 | 0.98 ± 0.95 | 2.91 ± 1.10 |
| Kinect | 0.58 ± 0.56 | 2.87 ± 1.97 | 5.61 ± 1.72 | 6.54 ± 2.04 |
| TRE 2 | | | | |
| P2000 | 0.72 ± 0.78 | 2.46 ± 1.12 | 1.12 ± 0.87 | 2.91 ± 1.37 |
| Kinect | 1.09 ± 0.49 | 3.19 ± 0.83 | 7.30 ± 1.19 | 8.11 ± 1.02 |
| TRE 3 | | | | |
| F200 | 0.83 ± 0.57 | 0.72 ± 0.52 | 1.40 ± 1.00 | 1.92 ± 0.98 |
| Kinect | 1.40 ± 0.65 | 1.97 ± 1.33 | 7.11 ± 1.33 | 7.60 ± 1.55 |

Values are reported as mean ± SD

Influence of Camera Choice

The calibration quality depends on the quality of the depth information. With the Intel RealSense camera, an average TRE of 2.58 mm can be achieved, while the calibration using the Microsoft Kinect® 360 (v1) achieves 7.42 mm due to poor depth quality and high errors along the z-axis.

Influence of Phantom Choice

Arbitrary objects can also be used as calibration phantoms. To validate this assumption, we used a stone and spine phantom for calibration and computed the TRE (Table 7). Theoretically, any physical object with an unambiguous 3D shape and a sufficient visibility in X-ray and infrared imaging can be used as calibration phantom. For instance, the spine phantom fulfills these requirements and the calibration results are relatively good. Due to some reflective proper-ties, the stone yields poor infrared imaging properties, and therefore, the calibration is of poor quality.

TABLE 7
The results of the TRE of arbitrary objects, where δx, δy, δz, and ||δ||2
are the Euclidean distances.

| TRE | δx | δy | δz | \|\|δ\|\|₂ |
|---|---|---|---|---|
| Spine | 1.03 ± 0.47 | 1.53 ± 1.27 | 1.88 ± 1.14 | 2.87 ± 1.28 |
| Stone | 1.00 ± 0.57 | 3.35 ± 2.39 | 2.46 ± 1.35 | 4.77 ± 2.05 |

Values are reported as mean ± SD

Mixed Reality Visualization

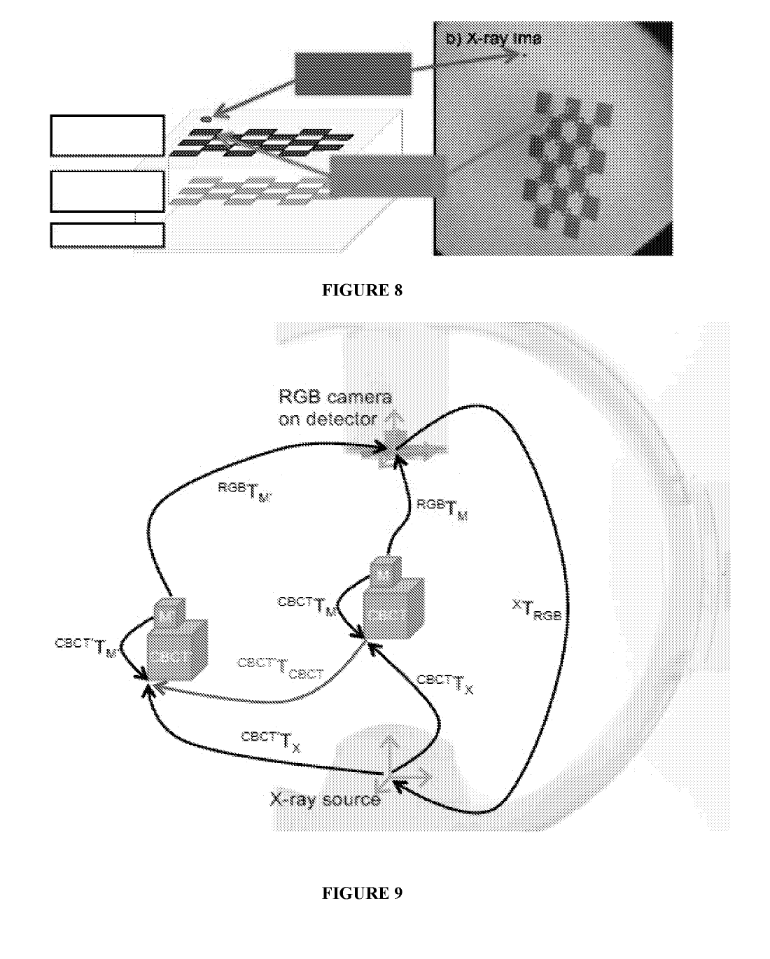

An example for inserting a guide wire into a spine phantom is shown in FIG. 8. This system could also be used for fast foreign body (shrapnel) removal.

Discussion and Conclusion

This paper proposes a novel methodology to calibrate an RGBD camera rigidly mounted on a C-arm and a CBCT volume. This combination enables intuitive intra-operative augmented reality visualization. The accuracy and repeatability of the algorithm are evaluated using several tests. Although the spatial resolution of the RGBD cameras in depth is poor (approximately ±5% of the depth), a reasonable registration accuracy of 2.58 mm is achieved. This paper has presented two applications with high clinical impact. First, image-guided drilling for cannulated sacral screw placement was demonstrated. Finally, the experiments are concluded with a simulated foreign body removal using shrapnel models.

To achieve the fused RGBD and DRR view, multiple steps are required. First, the CBCT and the patient's surface scans are acquired. The FPFH matching for fast initialization of ICP yields a robust and efficient calibration of data extracted from CBCT and RGBD. This enables the data overlay, resulting in an augmented reality scene. The calibration accuracy is strongly dependent on the quality of the depth information acquired from the RGBD camera.

In contrast to other calibration techniques, this method does not require a pre-defined marker or known 3D structure. Theoretically, the calibration technique functions with an arbitrary object for which the surface is visible in the CBCT and yields enough structural features. In a clinical scenario, a system would require a one-time calibration or at the discretion of the user.

The fusion of CBCT and RGBD into one common coordinate space enables several new concepts. First, any arbitrary view can be visualized as the spatial restrictions in terms of C-arm placement no longer apply. For instance, a view along the spine can be visualized while placing a Jamshidi needle. Secondly, the augmented reality scene can be viewed from different view-points simultaneously. This enables surgeons to align tools in all dimensions at the same time, possibly saving significant OR time.

Changes in the environment are currently not tracked. Moving the surgical table or RGBD camera may result in the loss of proper image alignment, which motivates further development of the CBCT and RGBD system. Beyond the aforementioned possibilities, the fusion of RGBD and CBCT could facilitate intra-operative surgical navigation as the RGBD camera could be used for tool or activity tracking. Understanding the activity would enable the automatic adjustment of the view in order to provide the most optimal view during interventions.

The proposed technique contributes to a novel calibration for RGBD and CBCT data and achieves an accuracy of 2.58 mm. This is promising for surgical applications, considering that validation X-ray images will remain part of the standard workflow. By acquiring more reliable depth information, this system could be later used for image-guided interventions to assist surgeons to perform more efficient procedures. The mixed reality visualization could enable an entire new field of novel applications for computer-aided orthopedic interventions.

### Example 4

### Automatic Intra-Operative Stitching of Non-Overlapping Cone-Beam CT Acquisitions

Summary

Cone-Beam Computed Tomography (CBCT) is one of the primary imaging modalities in radiation therapy, dentistry, and orthopedic interventions. While CBCT provides crucial intraoperative imaging, it is bounded by its limited imaging volume, resulting in reduced effectiveness in the OR. Therefore, orthopedic interventions, for instance, often rely on a large number of X-ray images to obtain anatomical information intraoperatively. Consequently, these procedures become both mentally and physically challenging for the surgeons due to excessive C-arm repositioning; and yet accurate 3D patient imaging is not part of the standard of interventional care. Our approach combines CBCT imaging with vision-based tracking to expand the image volume to increase the practical use of 3D intraoperative imaging. We attach a color and depth (RGBD) camera to a CBCT-enabled mobile C-arm, and co-register the video, infrared, and X-ray views. This permits the automatic alignment and stitching of multiple small, non-overlapping volumes to enable large 3D intraoperative imaging. Previous approaches of intraoperative image stitching mostly focused on 2D image mosaicing or parallax-free stitching, and relied on external tracking hardware, where our system is self-contained, and performs vision-based inside-out tracking to recover the spatial relationship between non-overlapping CBCT volumes. We propose and evaluate three methods to recover the relative motion of C-arm to patient, namely, visual marker tracking, surface tracking by fusing depth data to a single global surface model, and RGBD-based simultaneous localization and mapping. An extensive evaluation of each methodology is presented and compared to vision-based stitching of CBCT volumes and the state-of-the-art stitching methods. The experiments conducted on animal cadaver show sub-millimeter accuracy in the stitching of CBCT volumes.

Introduction

Intraoperative three-dimensional X-ray Cone-Beam Computed Tomography (CBCT) during orthopedic and trauma surgeries may yield the potential to reduce the need of revision surgeries and improve patient safety. Several works have emphasized on the advantages that C-arm CBCT offers for guidance in orthopedic procedures for head and neck surgery, Spine surgery, and K-wire placement in pelvic fractures. Other medical specialties, such as dentistry or radiation therapy, have reported similar benefits when using CBCT. However, commonly used CBCT devices exhibit a limited field of view of the projection images, and are constraint in their scanning motion. This results in a reduced effectiveness of the imaging modality in orthopedic interventions due to the small volume reconstructed.

For orthopedic traumatologists, achievement of correct length, alignment, and rotation of the affected extremity is the end goal of a fracture management strategy regardless of fixation technique. This can be difficult with the use of conventional fluoroscopy with limited field of view and lack of three-dimensional cues. For instance, it is estimated that malalignment (>5 degrees in the coronal or sagittal plane) is seen in approximately 10%, and malrotation (>15 degrees) in up to approximately 30% of femoral nailing cases. Cases involving minimally invasive techniques may face an even higher chance of malrotation.

Intraoperative stitching of two-dimensional fluoroscopic images has been investigated to address these issues. The value of stitched fluoroscopy images for orthopedic surgery was investigated. Radio-opaque referencing markers attached to the tool were used to perform the stitching. Trajectory visualization and total length measurement were the most frequent features used by the surgeons in the stitched view. The outcome was overall promising for future development, and the usability was counted as good. Similarly, X-ray translucent references were employed and positioned under the bone for 2D X-ray mosaicing. Alternatively, optical features acquired from an adjacent camera were used to recover the transformation. The aforementioned methods all benefit from external features for 2D mosaicing, thus do not require large overlaps. However, it remains a challenge to generalize these approaches to perform 3D volume stitching. Other overlap-independent stitching methods rely on prior models, which are mostly not available for injured patients in orthopedics and trauma interventions. Furthermore, while holding promise for length and alignment these systems provide little benefit over conventional methods concerning rotation. Additionally, these systems require images to be taken along the full length of the long bone in order to visualize the entire limb.

Orthopedics sports-related and adult reconstruction procedures could benefit as well. For example, high tibial and distal femoral osteotomies are utilized to shift contact forces in the knee in patients with unilateral knee osteoarthritis. These osteotomies rely on precise correction of the mechanical axis in order to achieve positive clinical results. Computer-aided navigation systems and optical trackers have shown to help achieve the desired correction in similar procedures; however, they carry limitations in terms of workflow requiring registration and skeletal fixed reference bases. Moreover, navigation systems are complex to setup, and rely on outdated patient data. Intraoperative CBCT has the potential to provide the advantage of a navigational system for osteotomies about the knee while fitting within the conventional surgical workflow.

In total hip arthroplasty, leg length discrepancy continues to be a significant complication and is a common reason for litigation against orthopedic surgeons. Many systems have been developed to address this issue including computer navigation and intraoperative two-dimensional fluoroscopy; however, they are not ideal. For example, intraoperative fluoroscopic views of the entire pelvis can be time consuming and difficult to obtain. Furthermore, intraoperative fluoroscopy can only be utilized for leg length determination for the anterior approach. Intraoperative CBCT could provide an alternative method for addressing leg length discrepancy for total hip arthroplasty while providing other advantages in terms of component placement.

The most promising case use is for mid comminuted fractures of the femur. Intraoperative three-dimensional X-ray CBCT yields the potential to address length, alignment, and rotation and to reduce the need for revision surgery due to malreduction. Known difficulties exist in addressing rotational alignment in mid-shaft comminuted femur fractures and the clinical impact of misalignment. Traditionally, to ensure proper femoral rotation, the contralateral leg is used as a reference. This procedures is described below. First, an AP radiograph of the contralateral hip is taken. This image is saved and particular attention is paid to anatomical landmarks such as how much lesser trochanter is visible along the medial side of the femur. Then the C-arm is translated distally to the knee. The C-arm is then rotated −0.90° to obtain a lateral radiograph of the healthy knee with the posterior condyles overlapping. These two images, the AP of the hip and lateral of the knee, determine the rotational alignment of the healthy side. To ensure correct rotational alignment of the injured side, an AP of the hip (on the injured side) is obtained, attempting to reproduce a similar AP radiograph to the contralateral side (a similar amount of lesser trochanter visible along the medial side of the femur). This ensures the position of both hips are similar. The C-arm is then moved distally to the knee of the injured femur and rotated −0.90° to a lateral view. This lateral image of the knee should match that of the healthy side. If they do not match, rotational correction of the femur can be performed, attempting to obtain a lateral radiograph of the knee on the injured side similar to that of the contralateral side.

In order to produce larger volumes, panoramic CBCT was proposed by stitching overlapping X-rays acquired from all the views around the interest organ. Reconstruction quality is ensured by introducing a sufficient amount of overlapping regions, which in return increases the X-ray dose. Moreover, the reconstructed volume is vulnerable to artifacts introduced by image stitching. An automatic 3D image stitching technique was proposed. Under the assumption that the orientational misalignment is negligible, and sub-volumes are only translated, the stitching is performed using phase correlation as a global similarity measure, and normalized cross correlation as the local cost. Sufficient overlaps are required to support this method. To reduce the X-ray exposure, previous studies incorporate prior knowledge from statistical shape models to perform a 3D reconstruction.

To optimally support the surgical intervention, our focus are CBCT alignment techniques that do not require the change of workflow or additional devices in the operating theater. Moreover, to avoid excessive radiation, we assume no overlap between CBCT volumes exist. These constraints motivate our work and lead to the development of novel methods, which are presented in this manuscript and compared to state of art solutions. We also discuss and compare the results of this work to the technique proposed in [30] as the first self-contained sys-tem for CBCT stitching. To avoid the introduction of additional devices, such as computer or camera carts, we propose to co-register the X-ray source, and a color and depth camera, and track the C-arm relative to the patient. This allows the mobile C-arm to remain self-contained, and independent of additional devices or the operating theater. Additionally, the imaging quality of each individual CBCT volume remains intact, and the radiation dose is linearly proportional to the size and numbers of the individual CBCT volumes.

Vision-Based Stitching Techniques for Non-Overlapping CBCT Volumes

To align and stitch non-overlapping CBCT volumes, the transformation between the individual volumes needs to be recovered. In this work, we assume only the relative relation-ship between CBCT volumes is of interest, and that the patient does not move during the CBCT scan. We exclude image-based solutions, as these are difficult or not applicable to non-overlapping regions of interest. Tracking systems introducing additional hardware in the surgical environment, such as external infrared tracking systems or other carts, disrupt the workflow and make the C-arm less mobile and more dependent on third-party equipment or the operating theater. Our tracking systems utilize a three-dimensional color camera, where depth (D) information is provided for each red, green, and blue (RGB) pixel. To keep the workflow disruption and limitation of free workspace minimal, we mount our RGBD camera near the image detector onto the mobile C-arm.

After a one-time calibration of the RGBD camera and X-ray source, the CBCT reconstruction is performed. Next, we use the calibration information and the 3D patient data and deploy several different tracking techniques.

RGB Camera, Depth Sensor, and X-Ray Source Calibration

The tracking of relative displacement of the patient requires the calibration of RGB camera, depth sensor, and X-ray source, resulting in three transformations. By means of reconstruction, the relationship between X-ray source and CBCT volume (CBCTTX) is known. To allow the X-ray source to remain under the surgical table, and to keep the surgical workflow intact, we mount the camera near the image detector.

To simultaneously calibrate the camera, depth sensor, X-ray source, and estimate the relationships between the three imaging devices, we designed a radiopaque checkerboard which can also be detected by the color camera and in the infrared image of the depth sensor. The checkerboard pattern shown in FIG. 8 comprises 5×6 fields, where every other one is black and backed by metal sheets. To calibrate the RGBD camera and depth sensor, we deploy a combination of automatic checkerboard detection and pose estimation and non-linear optimization to compensate for radial distortion, resulting in the camera projection matrices P_{RGB} and P_{D}. These projection matrices are optimized for all (n) valid checkerboard poses CBₙ. For each of the checkerboard poses we obtain the transformations from camera origin to checkerboard origin ^{CBn}T_{RGB}, and from depth sensor origin to checkerboard origin ^{CBn}T_{D}.

For X-ray images, the thin metal sheets cause a low contrast between the different checkerboard fields and the surrounding image intensities, which currently requires manual annotation of the outline of the checkerboard before the automatic detection can be deployed. After the automatic detection of the outlined checkerboard, the checkerboard poses ^{CBn}T_{X} and camera projection matrix P_{X} can be estimated similarly to an optical camera. In contrast to a standard camera, the X-ray imaging device provides flipped images to give the medical staff the impression that they are looking from the detector towards the sources. Therefore, the images are treated as if they were in a left-hand coordinate frame, and an additional pre-processing step or transformation needs to be deployed.

Finally, for each checkerboard pose CBn the RGB camera, depth sensor, and X-ray source poses are known, allowing for a simultaneous optimization of the three transformations RGB camera to X-ray source XTRGB, depth sensor to X-ray source XTD, and RGB camera to depth sensor DTRGB. This process concludes the one-time calibration step required at the time of the system setup.

CBCT Reconstruction

We hypnotized that the relationship between each X-ray projection image and the CBCT volume is known by means of reconstruction. To obtain the accurate and precise projection matrices, we perform a CBCT volume reconstruction and verify the X-ray source extrinsics by performing a 2D/3D registration. For each projection image acquired, the projection matrices are computed based on the known X-ray source geometry (intrinsics) and the orbital motion (extrinsics). Additionally, the spacing between the X-ray source positions is recorded by the mechanical C-arm encoders.

In some cases the orbital C-arm motion deviates from the assumed path, leading to erroneous projection matrices. Utilizing a simple 2D/3D registration based on Digitally Reconstructed Radiographs, Normalized Cross-Correlation (NCC) similarity cost, and Bound Constrained By Quadratic Approximation (BOBYQA) optimization, we verify the X-ray source extrinsics for each projection image. In case of significant deviation, the prior hypothesis of a known relationship between X-ray source origins and CBCT is not valid and requires the CBCT scan to be re-acquired.

Vision-Based Marker Tracking Techniques

After calibration of the cameras and X-ray source, the intrinsics and extrinsics of each imaging device are known. The calibration allows to track the patient using the RGB camera or depth sensor and apply this transformation to the CBCT volumes. This tracking technique relies on simple, flat markers with a high contrast pattern. They can be easily detected in an image, and the pose can be retrieved as the true size of the marker is known.

Visual Marker Tracking of Patient: The detection quality and tracking accuracy are best when the camera direction is perpendicular to the marker surface (camera angle 90°), and significantly decreases for shallow angles. To enable an overall good tracking accuracy, we deploy a multi-marker strategy and arrange markers on all sides of a cube, resulting in an increased robustness and pose-estimation accuracy. The marker cube is rigidly attached to the phantom or anatomy of interest.

After performing the orbital rotation and acquiring the projection images for the reconstruction of the first CBCT volume, the C-arm is rotated to a pose R for which the projection matrix PR is known (see Sec. II-A), and the transformation from X-ray source origin to CBCT origin is denoted CBCTTX. Ideally, this pose is chosen to provide an optimal view of relative displacement of the marker cube, as the markers are tracked based on the color camera view. The center of the first CBCT volume is defined to be the world origin, and the marker cube M can be represented in this coordinate frame based on the camera to X-ray source calibration:

^{CBCT}T_{M}=^{CBCT}T_{X}^{X}T_{RGB}·^{RGB}T_{M}. (1)

The transformations are depicted in FIG. 4. The patient, surgical table, or C-arm are re-positioned in order to acquire the second CBCT volume. During this movement, the scene and the marker cube are observed using the color camera, allowing the computation of the new pose of the marker cube RGBTM′. Under the assumption that the relationship between CBCT volume and marker (Eq. 1) did not change, the relative displacement of the CBCT volume is expressed as a new transformation from X-ray source to new CBCT volume:

^{CBCT′}T_{X}=^{CBCT}T_{M}^{RGB}T_{M}^{-1x}T_{RGB}⁻¹ (2)

Hence, the relative CBCT displacement is derived as:

^{CBCT′T=CBCT T}_{M}^{RGB T}_{M}^{-1xT}_{RGB}⁻¹ (3)

Visual Marker Tracking of Surgical Table: In many orthopedic interventions, the C-arm is used to validate the reduction of complex fractures. This is mostly done by moving the C-arm rather than the injured patient. We can therefore hypothesize that the patient remains on the surgical table and only the relationship between table and C-arm is of interest, which has also been assumed in previous work.

A pre-defined array of marker is mounted in the bottom of the surgical table, which allows the estimation of the pose of the C-arm relative to the table.

RGBD Simultaneous Localization and Mapping for Tracking

RGBD devices are ranging cameras which allow the fusion of color images and depth information. These cameras enable the scale recovery of visual features using depth information from a co-calibrated IR sensor. We aim at using RGB and depth channels concurrently to track the displacement of patient relative to a C-arm during multiple CBCT acquisitions.

Simultaneous Localization and Mapping (SLAM) has been used in the past few decades to recover the pose of a sensor in an unknown environment. The underlying method in SLAM is the simultaneous estimation of the pose of perceived landmarks, and updating the position of a sensing device. A range measurement device such as a laser scanner is mostly used together with a moving sensor (e.g. mobile robot) to recover the unknown scales for features and the translational components. An RGBD SLAM was introduced in a previous study where the visual features are extracted from 2D frames, and later the depth associated to those features are computed from the depth sensor in the RGBD camera. These 3D features are then used to initialize the RANdom SAmple Consensus (RANSAC) method to estimate the relative poses of the sensor by fitting a 6 DOF rigid transformation.

RGBD SLAM enables the recovery of the camera trajectory in an arbitrary environment using no prior models, as well as incrementally creating a global 3D map of the scene in real-time. We assume that the global 3D map is rigidly connected to the CBCT volume, which allows the computation of the relative volume displacement analog to the technique presented in Vision-based Marker Tracking Techniques.

Surface Reconstruction and Tracking Using Depth Information

Surface information obtained from the depth sensor in an RGBD camera can be used to reconstruct the patient's surface, which simultaneously enables the estimation of the sensor trajectory. Kinect Fusion® enables a dense surface reconstruction of a complex environment and estimates the pose of the sensor in real-time.

Our goal is to use the depth camera view and observe the displacement, track the scene, and consequently compute the relative movement between the CBCT volumes. This tracking method involves no markers, and the surgical site is used as reference (real-surgery condition). To utilize the RGBD camera together with the C-arm, the stereo relation of the depth camera needs to be known with respect to the C-arm X-ray source. This calibration step is discussed in RGB Camera, Depth Sensor, and X-ray Source Calibration.

Kinect Fusion® relies on a multi-scale Iterative Closest Point (ICP) between the current measurement of the depth sensor and a globally fused model. The ICP incorporates large number of points in the foreground, as well as the background. Therefore, a moving object with a static background causes unreliable tracking. Thus, multiple non-overlapping CBCT volumes are only acquired by repositioning the C-arm instead of the surgical table.

Similar transformations shown in FIG. 9 are used to compute the relative CBCT displacement CBCT′TCBCT, where D defines the depth coordinate frame and D′TD is the relative camera pose computed using Kinect Fusion®:

^{CBCT′}T_{CBCT}=^{CBCT}T_{D}^{D}T_{D}⁻_{CBCT}T_{D}⁻¹ (4)

Related Work as Reference Techniques

The use of external infrared tracking systems to observe the displacement of patients or the X-ray machine are widely accepted in clinical practice, and are usually not deployed to automatically align and stitch multiple CBCT volumes. A major disadvantage of external tracking systems is the introduction of additional hardware to the operating room, and the accumulation of tracking errors when tracking both the patient and C-arm. However, to provide a reasonable reference to our vision-based tracking techniques, we establish the methodology to use an infrared tracking system to perform CBCT volume stitching (Infrared Tracking System). This chapter concludes with an brief overview of our previously published vision-based stitching technique (Two-dimensional Feature Tracking) and it is put in context of our chain of transformations.

Infrared Tracking System

Using a passive infrared tracking system, objects that should be tracked need to be equipped with highly infrared reflective markers, which comprise a unique combination of three or more reflective spheres. This enables the computation of the precise pose of the marker. For surgical tracking, the marker is usually rigidly screwed to a bone, or attached to the patient's surface in close proximity to the region of interest. In the following we first discuss the calibration of C-arm to the CBCT coordinate frame using IR markers mounted on the C-arm, and subsequently the C-arm to patient tracking using this calibration.

Calibration: This step includes attaching markers to the C-arm and calibrating them to the CBCT coordinate frame. This calibration later allows us to close the patient, CBCT, and C-arm transformation loop and perform reliable tracking of relative displacement. The spatial relation of the markers on the C-arm with respect to the CBCT coordinate frame is illustrated in FIG. 5 and is defined as:

^{CBCT′}T_{Carm}=^{CBCT}T_{IR}^{Carm}T_{D}⁻¹ (5)

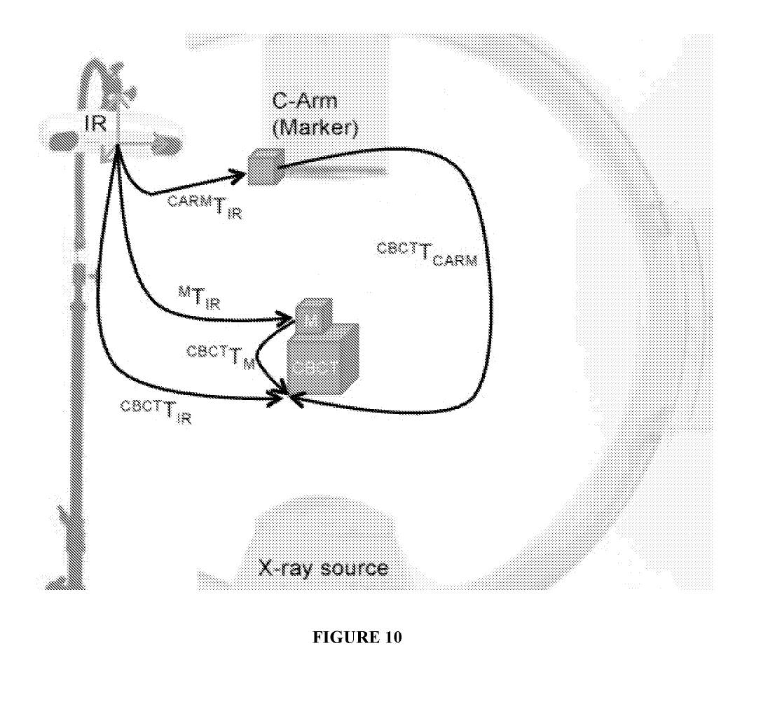

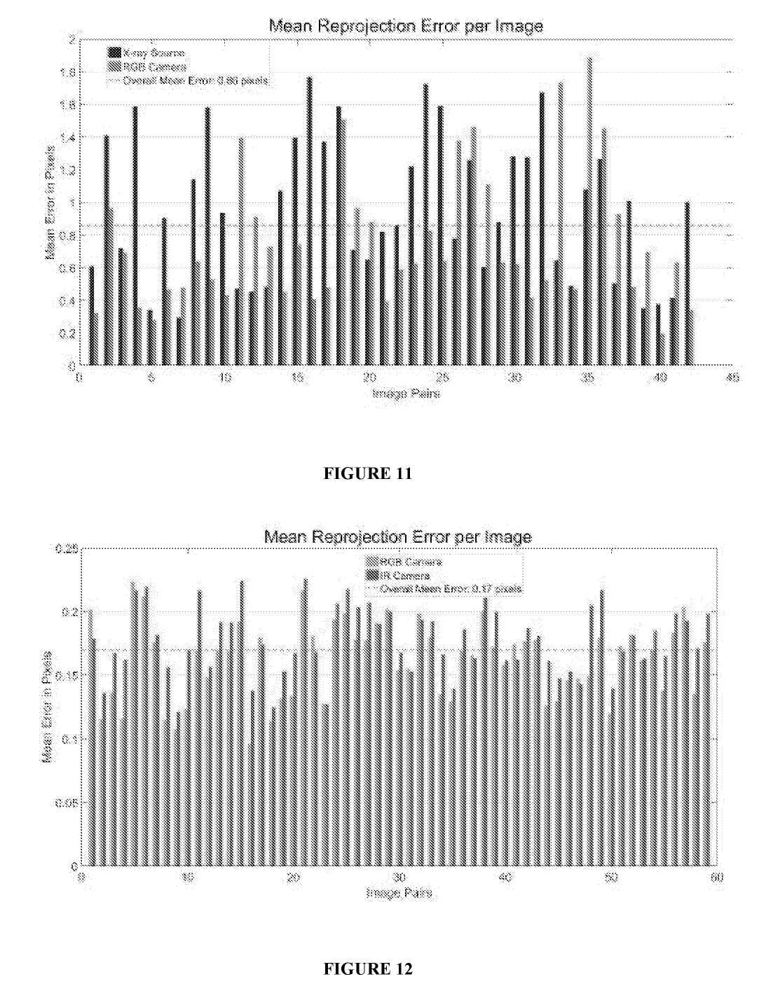

First step in solving Eq. (5) is to compute CBCTTIR. This estimation requires at least three marker positions in both CBCT and IR coordinate frames. Thus, a CBCT scan of another set of markers (M in FIG. 10) is acquired and the spherical markers are located in the CBCT volume. Here, we attempt to directly localize the spherical markers in the CBCT image instead of X-ray projections. To this end, a bilateral filer is applied to the CBCT image to remove the noise while preserving the edges. Next, the weak edges are removed by thresholding the gradient of the CBCT, and the strong edges corresponding to the surface points on the spheres are preserved. The resulting points are clustered into three partitions (one cluster per sphere), and the centroid of each cluster is computed. Then an exhaustive search is performed in the neighborhood around the centroid with the radius of ±(r+δ), where r is the sphere radius (6.00 mm) and δ is the uncertainty range (2.00 mm). The sphere center is located by a least-square minimization using its parametric model. Since the sphere size is provided by the manufacturer and is known, we avoid using classic RANSAC or Hough-like methods as they also optimize over the sphere radius.

Next step is to use these marker positions in CBCT and the IR tracker frame and compute the CBCTTIR. For this we use the method previously suggested, where two points sets are translated to the origin. The rotational components are recovered using the SVD of the correlation matrix between the point sets, and then the closed-form translation is computed as the Euclidean distance of the two point sets considering the optimal rotation. Consequently, we can close the calibration loop and solve Eq. (5) using CBCTTIR, and CarmTIR which is directly measured from the IR tracker.

Tracking: The tracking stream provided for each marker configuration allows the computation of the patient motion. After the first CBCT volume is acquired, the relative patient displacement is estimated before the next CBCT scan is performed.

Considering the case where the C-arm is re-positioned (from Carm to Carm′ coordinate frame) to acquire CBCT volumes (CBCT and CBCT′ coordinate frames), and the patient is fixed on the surgical table, the relative transformation from IR tracker to CBCT is defined as following:

^{CBCT′}T_{IR}=^{CBCT}TCarm^{Carm}T_{IR} (6)

^{CBCT′}T_{IR}=^{CBCT}TCarm^{Carm}T_{IR} (7)

The relation between the C-arm and the CBCT is fixed, hence CBCTTCarm=CBCT′TCarm′. Furthermore, the relative transformation from CBCT to CBCT′ is given by the following equation:

^{CBCT′}T_{CBCT}=^{CBCT}T_{IR}^{CBCT}T_{IR}⁻¹ (8)

In order to consider patient movement, markers (coordinate frame M in FIG. 5) may also be attached to patient (screwed into the bone), and be tracked in the IR tracker coordinate frame. CBCTTM is then defined as:

₁^{CBCT′}T_{M}=^{CBCT}T_{Carm}^{Carm}T_{IR}^{-M}T_{IR}⁻ (9)

Assuming the transformation between CBCT and marker is fixed during the intervention (CBCT′TM′=CBCTTM) and combining Eq. 6, Eq. 7 with Eq. 9, volume poses in the tracker coordinate frame are defined as:

^{CBCT′}T_{IR}=^{CBCT}T_{M}^{M}T_{IR} (10)

^{CBCT′}T_{IR}=^{CBCT}T_{M}^{M}T_{IR} (11)

solving Eqs.(10) and (11) leads to recovery of CBCT displacement using Eq. (8).

Two-Dimensional Feature Tracking

We use the camera attached to the mobile C-arm, and the positioning laser in the base of the C-arm to recover the 3D depth scales, and consequently stitched the sub-volumes as presented previously. The positioning-laser in the base of the C-arm spans a plane which intersects with the unknown patient surface. The laser line can be observed as a curve in the camera image, and used to approximate the scale of features nearby.

Calibration: To determine the relationship between camera and laser plane, we perform a calibration using multiple checkerboard poses. At each of the n poses the laser intersects the origin of the checkerboard, which allows to recover points on the laser plane in the camera coordinate frame. By per-forming RANSAC-based plane fitting, the plane coefficients are computed. As presented previously, some checkerboard poses are treated as outliers and rejected.

Tracking: Following a previous technique, the tracking algorithm comprises following steps: (i) Automatic detection of Speeded Up Robust Features (SURF) in every frame; (ii) Matching features from one frame to the next, and rejecting outliers by estimating the Fundamental Matrix; (iii) Automatically detecting the laser line and computing the 3D shape based on the known laser plane; (iv) Recovering the scale of the features using the scale of the nearby laser line; (v) Estimating the 3D transformation for the sets of 3D features; and (vi) Validating transformation estimation by applying to 3D laser line. Finally, the frame-by-frame transformations are accumulated and result in an transformation CBCT′TCBCT.

Experiments and Results

As motivated in the introduction, intra-operative, user-friendly alignment and stitching of multiple CBCT volumes may yield a significant clinical impact; yet, previous publications were mostly limited to stitching of 2D images. In this section, we report the results of the vision-based methods to stitch multiple CBCT volumes as presented in VISION-BASED STITCHING TECHNIQUES FOR NON-OVERLAPPING CBCT VOLUMES. The same experiments are preformed using the methods outlined in RELATED WORK AS REFERENCE TECHNIQUES, namely using a commercially available infrared tracking system, and our previously published techniques.

This section is structured as following: First, we introduce the system setup and data acquisition. Next we report the results for the calibration of RGB camera, infrared camera, and X-ray source. Finally, we present the results of the vision-based tracking techniques in phantom and animal cadaver experiments.

Experimental Setup and Data Acquisition

Experimental Setup: The CBCT-enabled motorized C-arm is positioned relative to the patient by utilizing the positioning-lasers, which are built into the image intensifier and C-arm base. In contrast to existing techniques we do not require additional hardware setup around the C-arm, but we attach a camera near the detector plane of the C-arm in such manner that it does not obstruct the surgeons access to the patient. Compared to the Camera-Augmented Mobile C-arm, our system does not require a mirror construction and consequently will not reduce the free space (area between the image intensifier and the X-ray tube). Hence, it can better accommodate obese patients and provides more operating space.

Our system is composed of a mobile C-arm, ARCADIS® Orbic 3D, from Siemens Medical Solutions and an Intel Realsense® SR300 RGBD camera, Intel Corporation. The SR300 is relatively small (X=110.0±0.2 mm, Y=12.6±0.1 mm, Z=3.8-4.1 mm), and integrates a full-HD RGB camera, and an infrared projector and infrared camera, which enable the computation of depth maps. The SR300 is designed for shorter ranges from 0.2 m to 1.2 m for indoor uses. Access to raw RGB and infrared data are possible using the Intel RealSense SDK. The C-arm is connected via Ethernet to the computer for CBCT data transfer, and the RGBD camera is connected via powered USB 3.0 for real-time frame capturing.

CBCT Volume and Video Acquisition: To acquire a CBCT volume, the patient is positioned under guidance of the lasers. Then, the motorized C-arm orbits 190° around the center visualized by the laser lines, and automatically acquires a total of 100 2D X-ray images. Reconstruction is performed using an MLEM-based ART method, resulting in a cubic volume with a 512 voxels along each axis and an isometric resolution of 0.2475 mm. For the purpose of reconstruction, we use the following geometrical parameters provided by the manufacturer: source-to-detector distance: 980.00 mm, source-iso-center distance: 600.00 mm, angle range: 190°, detector size: 230.00 mm×230.00 mm.

During the re-arrangement of C-arm and patient for the next CBCT acquisition, the vision-based tracking results are recorded. For this re-arrangement we consider the clinically realistic scenario of a moving C-arm and a static patient. However, as extensively discussed in Sec. II, for marker-based methods only the relative movement of patient to C-arm is recorded, hence there are no limitations on allowed motions.

Calibration Results

Vision-based stitching requires a precise calibration of RGB camera, infrared camera, and X-ray source. This is achieved using a multi-modal checkerboard (see FIG. 8), which is observed at multiple poses using the RGB camera and depth sensor. In addition, at each of these poses, an X-ray image is acquired. We use these images to perform the stereo calibration for the RGB-to-X-ray and RGB-to-infrared. An asymmetric checkerboard pattern is designed by first printing a black-and-white checkerboard pattern on a paper. Thin metal squares are then carefully glued to the opposite side of the printed pattern in order to make a radio-opaque checkerboard. The black-and-white pattern is detected in the RGB and infrared camera views, and the radio-opaque pattern is visible in the X-ray image. We use a 5×6 checkerboard where each square has a dimension of 12.655 mm. The distance between the black-and-white and the radio-opaque metal pattern is negligible. Thus, for the purpose of stereo calibration we assume all three cameras (RGB and infrared cameras, and X-ray source) observe the same pattern.

Due to the inherent noise in the X-ray images (random noise, Poisson noise, or other noises caused by radiation scattering), we define the four outer corners of the calibration pattern manually. This limits the search for other corner points to the area defined by these outer points, and reduces false-positive detection. Every other corner point is then detected automatically using a previously described calibration toolbox. 72 image triplets (RGB, infrared, and X-ray images) were recorded for the stereo calibration. Images with high reprojection errors or significant motion blurring artifacts were discarded from this list for a more accurate stereo calibration. The stereo calibration between the X-ray source and the RGB camera was eventually performed using 42 image pairs with the overall mean error of 0.86 pixels (FIG. 11). The RGB and infrared cameras were calibrated using 59 image pairs, and the overall reprojection error is 0.17 pixels (FIG. 12).

Experiments and Results

Our vision-based tracking methods are all tested and evaluated on an animal cadaver (pig femur). For these experiments, we performed the non-overlapping stitching of CBCT volumes with each method individually under realistic OR conditions. Subsequently, we measured the absolute distance between the implanted landmarks inside the animal cadaver and compared the results to a ground-truth acquired from a CT scan. The outcome of these experiments are compared to an Infrared-based tracking approach (baseline method) as well as the laser-guided tracking method. The results are reported in Table 8. The lowest tracking error is 0.33±0.30 mm and is achieved by tracking AR tags attached to the patient. Aligning and stitching volumes based on visual marker tracking or RGBD-SLAM has sub-millimeter error, while tracking by only the use of depth results in higher error (˜1.72 mm). The alignment of CBCT volumes using an Infrared tracker, or using two-dimensional color features also have errors larger than a millimeter.

In Table 8 we also report the angles between the mechanical and the anatomical axis of the femur (Tibio Femoral Angle), as well as the angle between the mechanical axis and the knee joint line (Lateral-distal Femoral Angle) using the vision-based stitching methods. The results indicate minute variations among different methods.

TABLE 8

| | | | | | Lateral- |
|---|---|---|---|---|---|
| | Stitching | Standard | Absolute | Tibio | distal |
| Tracking | Error | Deviation | Distance | Femoral | Femoral |
| Method | (mm) | (mm) | Error (%) | Angle (°) | Angle (°) |
| Measurement | — | — | — | 7.1° | 81.7° |
| in CT | | | | | |
| Infrared | 1.64 | 0.87 | 0.73 | 7.4° | 84.1° |
| Tracking | | | | | |
| Two- | 1.18 | 0.28 | 0.62 | — | — |
| dimensional | | | | | |
| Feature | | | | | |
| Tracking [30] | | | | | |
| Visual Marker | 0.33 | 0.39 | 0.14 | 7.7° | 81.2° |
| Tracking of | | | | | |
| Patient | | | | | |
| Visual Marker | 0.62 | 0.21 | 0.26 | 6.4° | 83.9° |
| Tracking of | | | | | |
| Surgical Table | | | | | |
| RGBD-SLAM | 0.91 | 0.59 | 0.42 | 6.6° | 82.3° |
| Tracking | | | | | |
| Tracking | 1.72 | 0.72 | 0.79 | 8.1° | 84.8° |
| Using Depth | | | | | |
| Information | | | | | |

ERRORS ARE COMPUTED BY MEASURING THE AVERAGE OF THE ABSOLUTE DISTANCES BETWEEN 8 RADIOLUCENT LANDMARKS IMPLANTED INTO THE FEMUR HEAD, GREATER TROCHANTER, PATELLA, AND THE CONDYLE. THE RESIDUAL DISTANCES ARE MEASURED BETWEEN THE OPPOSITE SIDES OF THE FEMUR (FROM HIP TO KNEE). EACH METHOD IS TESTED TWICE ON THE ANIMAL CADAVER. FIRST TWO ROWS CORRESPOND TO THE REFERENCE METHODS, AND THE FINAL FOUR ROWS PRESENT THE RESULTS USING VISION-BASED METHODS SUGGESTED IN THIS PAPER.

Discussion and Conclusion

We presented several vision-based techniques to stitch non-overlapping CBCT volumes intraoperatively and compared them to related work. Our system design allows tracking of patient or C-arm movement with minimal increase of workflow complexity and without introduction of external tracking systems.

We attached an RGB and depth camera to a mobile C-arm, and deployed state-of-art computer vision techniques, and consequently stitched the sub-volumes. As a result of this method, the stitching is performed with low dose radiation, linearly proportional to the size of non-overlapping sub-volumes. As motivated in the introduction, we expect this to be applicable to intraoperative planning and validation for long bone fracture or joint replacement interventions, where multi-axis alignment and absolute distances are difficult to visualize and measure from the 2D X-ray views. In this work, we target cases with large gaps between the volumes and focus our approach on recovering the spatial relationship of separated regions of interest.

In this paper, we presented three vision-based tracking methods for non-overlapping CBCT volume stitching, namely, visual marker tracking, surface tracking by fusing depth data to a single global surface model, and RGBD-based Simultaneous Localization and Mapping (SLAM). These approaches estimate the relative CBCT volume displacement based on only RGB, only depth, or a combination of RGB and depth information. Utilizing the stitching techniques presented in this work, the image quality remains intact, and the radiation dose is linearly proportional to the size of the individual non-overlapping sub-volumes.

We performed the validation experiments on an animal cadaver, and compared the stitching outcome to an infrared tracking system, as well as prior work. In these experiments we used a CT scan of the animal cadaver as the ground truth data. The visual marker-based tracking achieved the lowest tracking error (0.33 mm) among all methods. The high accuracy is due to utilizing a multi-marker strategy which avoids tracking in shallow angles. Stitching solely based on depth information has 1.72 mm error, and the tracking with RGB and depth information together has 0.91 mm error. In a clinically realistic scenario, the surgical site comprises drapes, blood, exposed anatomy, and surgical tools which allows the extraction of large number of useful color features in a color image. The authors believe that a marker-less RGBD¬SLAM stitching system can use the aforementioned color information, as well as the depth information from the co-calibrated depth camera, and provide reliable CBCT image stitching for orthopedic interventions.

Prior method for stitching of CBCT volumes involves the use of an RGB camera attached near the X-ray source. This method uses the positioning-laser on the C-arm base to recover the depth of the visual features detected in the RGB view. Therefore, all image features are approximated to be in the same depth scale from the camera base. Hence, very limited number of features close to the laser line are used for tracking. This will contribute to poor tracking when the C-arm is rotated as well as translated. In this work we also avoided stitching of projection images due to the potential parallax effect. In a ruler-based stitching of projections, since the ruler plane is different from the stitching plane the parallax effect occurs. Parallax effect causes incorrect stitching and the length and angles between the anatomical landmarks will not be preserved in the stitched volume.

The benefits of using cameras with a C-arm for radiation and patients safety, scene observation, and augmented reality has been emphasized in the past. This work presents a 3D/3D intraoperative image stitching technique using a similar opto X-ray imaging system. Our approach does not limit the working space, nor does it require any additional hardware besides one RGBD camera near the image intensifier. The C-arm remains mobile, self-contained, and independent of the operating room.

### Example 5

### Simultaneous Segmentation, Reconstruction and Tracking of Surgical Tools in Computer Assisted Orthopedics Surgery

Summary

Purpose

Several complex orthopedics interventions would possibly benefit from an intuitive, real-time, and simple tracking technique to assist the surgeon during an image-guided interventions. This paper presents an integration of a tracking component into an advanced visualization environment. Consequently, this enables an intuitive guidance by supporting the surgeon with aligning surgical tools with the planning data, and enabling an easy and fast localization of the starting point.

Methods

For both visualization of the surgical site and tracking the surgical tools we use an RGBD camera attached to the mobile C-arm. The real-time model-based tracking is based on the depth data from the RGBD camera. The tracking algorithm automatically segments parts of the object using RGBD images, reconstructs the model frame by frame, and compares it with the tool model to recover its current position. The orientation is estimated and visualized together with the pre-operatively planned trajectory. The tracked surgical tool and planned trajec-tory are overlaid on the medical images, such as X-ray and CBCT volumes.

Results

The tracking and system accuracy is evaluated experimentally by targeting radiopaque markers using the tracked surgical instrument. Additionally, the orientation is evaluated by aligning the tool with planned trajectory. The error is computed in terms of target registration error and distance from the expected paths. When large parts of the surgical instrument are occluded by the clinician's hand, our algorithm achieves an average error as low as 3.04 mm, while the error is reduced to 1.36 mm when fewer occlusions are present.

Conclusion

The system allows the surgeon to get close to their planned trajectory without using additional X-ray images in a short time, and, thereafter, use few X-ray images to confirm the final alignment before insertion. The real-time RGBD data enables live feedback of the tool's orientation comparing with the planned trajectory, which provides an intuitive understanding of the surgical site relative to the medical data.

Introduction

Mobile C-arms are frequently used in minimally invasive orthopedic and trauma interventions, and provide intra-operative X-ray and Cone-Beam CT (CBCT) imaging. The intra-operative medical images result in reduction of blood loss, collateral tissue damage, and the total operation time. However, mental mapping between 2D X-ray images, the patient's body, and surgical tools remains a challenge. Typically, the correct placement of each k-wire/screw requires numerous X-ray images and may be preceded by several failed attempts until the target is reached from a correct orientation. This leads to an endangerment of the patient's safety, may result in high X-ray radiation exposure of the patient and the surgical staff, increases the overall OR time, and frustrates the surgical team.

Surgical navigation systems are used to track tools and the patient with respect to the medical images, and therefore assist the surgeons with their mental alignment and localization. These systems are mainly based on outside-in tracking of optical markers on the C-arm and recovering the spatial transformation between the patient, medical images, and the surgical tool. The modern navigation systems reach a sub-millimeter accuracy However, they have no significant influence on the OR time reduction, but rather require a cumbersome pre-operative calibration, occupy extra space, and suffer from line-of-sight limitation. Last but not least, navigation is mostly computed based on pre-operative and outdated patient data. Thus, deformations and displacements of the patient's anatomy are not considered.

Alternative solutions attach cameras to the C-arm, and co-register them with the X-ray image. The camera is mounted near the X-ray source, and by utilizing an X-ray transparent mirror, the camera and X-ray views would be similar. Therefore, the camera and X-ray origins are aligned, and they remain calibrated due to the rigid construction. To overlay undistorted and semi-translucent X-ray images onto the live video camera, optical and radiopaque markers on a calibration phantom are detected and aligned. The Augmented Reality (AR) provides an intuitive visualization of the X-rays and a live optical view of the surgical site.

These system were tested during 40 orthopedic and trauma surgeries, and demonstrated promising improvement in X-ray dose reduction and localization in the 2D plane perpendicular to the X-ray view. However, this technique requires the introduction of large spherical markers and the medical imaging is limited to 2D X-ray images co-registered to the view of the optical camera. Furthermore, this requires the X-ray source to be positioned above the patient rather than under the table, which reduces the surgeon work space, and increases scatter radiation to the clinical staff. In a previous study, an Red-Green-Blue-Depth (RGBD) camera was mounted on a mobile C-arm, and calibrated with X-Ray. However, no contribution towards tool tracking, or simplification of surgery was presented.

Vision-based tracking of marker and a simple AR visualization combining Digitally Reconstructed Radiographs (DRR) with live video from a stereo camera attached to the detector plane of the C-arm was presented previously. Using a hex-face optical and radiopaque calibration phantom, paired-point registration is per-formed to recover the relationship between X-ray image and camera origin. The vision-based tracking requires visual markers and the stereo camera on the C-arm. This work takes a step further from static 2D-2D visualization to interactive 2D-3D visualization. However, tracking for an image-guided navigation requires the introduction of marker on the surgical instrument.

Several work suggested external optical navigation systems for tracking surgical tools and medical instruments during the intervention. These methods provide sub-millimeter accuracy, but disrupt the workflow and induce line-of-sight issues. An alternative solution in C-arm guided intervention is to track the tools with fluoroscopic images. Image processing is done on fluoroscopic images to extract the tools and thus determine the tool position and orientations. This approach does not have line-of-sight problems, however, increases the radiation exposures. On the other hand, in computer vision industry, marker-less tracking of rigid objects by incorporating an RGBD camera has previously been performed using Iterative Closest Point (ICP) algorithm. A 3D user-interface application based on this method is introduced, which allows 3D tracking of an object held by the user. This work assumes a static background, and the tracking requires the generation of user-specific hand models for tracking the objects occluded by hands. This enlightens an idea of using 3D sensing for tool tracking, which requires no makers nor extra fluoroscopic images.

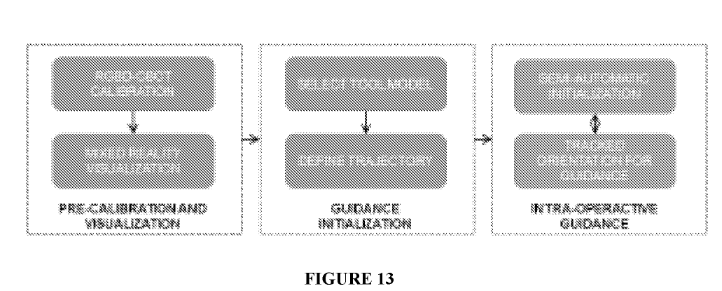

We propose a real-time tracking of the surgical tool exclusively using RGBD data and virtual models. No visual or other physical markers are introduced. The suggested workflow is illustrated in FIG. 13. The CBCT device and RGBD sensor are pre-calibrated, which allows data fusion between their data for a mixed reality visualization. At the beginning of the intervention, surgeons select the surgical tool model and define the expected trajectory on the CBCT or CT data. During the intervention, we use the tracking information and augment this virtual model at the position of the drill inside the mixed reality environment. When the drill is sufficiently close to the initialization, the tracking will automatically start. There-after, surgeons can interact with the scene and align the tracked tool's orientation with the planned trajectory. Lastly, a few X-ray images are acquired from different view-points to ensure the correct alignment and compensate for minute tracking errors.

In this paper we present modification of advanced computer vision algorithms for surgical tool tracking in the presence of occlusions. Additionally, we combine our tracking technique with a mixed reality visualization, which enables the simultaneous and real-time display of patient's surface and anatomy (CBCT or X-ray), the surgical site, and objects within the surgical site (clinician's hand, tools, etc.). This unique and intuitive mixed reality scene provides guidance and support for fast entry point localization. Without taking any X-rays images, the system enables surgeons to quickly align the tracked surgical tool's orientation with the planned trajectory or anatomy of interest. This can thereby shorten the operation time, and reduce radiation exposure.

Materials and Methods

Our technique requires an RGBD camera to be installed on the gantry of the mobile C-arm, preferably near the detector to remain above the surgical site during the intervention. In this section, we present a calibration technique for RGBD camera and mobile C-arm, the marker-less tool tracking algorithm, and the integration of tool tracking and mixed reality visualization.

Calibration of RGBD Camera and Mobile C-Arm

The calibration is performed by obtaining an RGBD and CBCT scan from a radiopaque and infrared-visible calibration phantom.

After surface extraction, the meshes are pre-processed to remove outliers and noise. The set of points are PDEPTH and PCBCT. The surfaces are registered using the SAmple Consensus Initial Alignment (SAC-IA) with Fast Point Feature Histogram (FPFH). This method provides a fast and reliable initialization TO, which is then used for the Iterative Closest Points (ICP) algorithm to complete the final calibration result:

$${}_{}^{DEPTH}{T}{}_{CBCT}^{} = {min}_{T} {\sum }_{i , j} {‖ {p}_{i}^{DEPTH} - {Tp}_{j}^{CBCT} ‖}_{2}^{2} \qquad ( 7 )$$

The resulting transformation PDEPTH and PCBCT allows the fusion of CBCT and RGBD information in a common coordinate frame. This enables tool tracking relative to anatomical structures, and mixed reality visualization.

Real-Time Tool Tracking Using Incremental Segmentation on Dense SLAM

Tool tracking is carried out by registering surface segments from the 3D tool model to automatically extracted surface segments in each frame using ICP. Nevertheless, for the specific application at hand, it has to be considered that the presence of a hand holding the drill causes severe occlusions that affect tracking convergence and accuracy. To deal with these limitations, we propose to exploit the automatic 3D surface segmentation to only track the segment corresponding to the drill and, therefore, removing possible outliers during the ICP stage.

The depth map is automatically segmented based on the angles between the surface normals, resulting in smooth surface segments. Among these segments, we detect the segments that correspond to the 3D tool model by comparing them (via 3D overlap) with the visible part of the 3D tool model. The visible part is computed by rendering a virtual view using the camera pose estimated in the previous frame. All segments of the depth map, which yield a 3D overlap higher than a threshold with the visible tool model, are merged into a set of Tool Segments (TS). It is the subset of points from the depth map to be used during ICP, which is carried out with a point-to-plane error metric to estimate the current pose of the tool. Correspondences between the points in the TS and the 3D tool model are obtained by projecting the current visible part of the tool model to the TS. The use of a limited subset of points belonging to the tool surface allows not only to better deal with occlusion, but also to track the drill in view of the camera.

To improve robustness towards occlusion, in addition to model-to-frame tracking, we also deploy frame-to-frame temporal tracking by adding to the ICP correspondences previously obtained also those provided by overlapping 3D segments between the TS in the previous frame and the TS in the current frame. Hence, the merged set of correspondences is jointly used to minimize the registration residual. This is particularly useful in presence of high level of occlusion, where the 3D surface of the hand holding the tool is deployed to robustly estimate the current camera pose with respect to the tool. The tracking quality is used to compute the mean residual between the points in current depth map and the surface of tool model. When this mean residual error exceeds a certain threshold, the tracking needs to be considered to be failed.

3D Augmented Mobile C-Arm Visualization with Marker-Less Tool Tracking

We generate a mixed reality scene using a CBCT volume, and the real-time RGBD information, and incorporate the tracking information by rendering a virtual model of the surgical instrument within this environment. The user can position multiple virtual cameras in this scene to view the anatomy and tools from multiple desired views.

This system design has several advantages: Firstly, it uses multiple virtual views, which are not limited to the actual camera or tracker position. This allows views even from within the patient's anatomy. Furthermore, users can observe multiple desired views at the same time, which greatly helps depth perception. As evaluated previously, the system significantly reduces the radiation use, reduces surgical staff workload, and shortens the operation time. Secondly, it performs marker-less tracking of any arbitrary surgical tool (given a model), which is partially visible to the RGBD camera. It reduces the line-of-sight problem as the camera is now placed in the immediate environment of the user, and provides reasonable tracking accuracy. Lastly, the interaction of the tracked tools in the multi-view visualization allows users to perceive the target depth, orientation, and their relationship intuitively and quickly. Together with the overlaid of planned trajectory and the estimated tool's orientation, it simplifies the complicated k-wire/screw placement procedure, which typically requires numerous X-ray images. Our system allows the user to achieve a nearly correct alignment very fast (within a minute).

Experimental Validation and Results

System Setup: The system comprises a Siemens ARCADIS® Orbic 3D C-arm (Siemens Healthineers, Erlangen, Germany) and Intel RealSense® SR300 RGBD camera (Intel Corporation, Santa Clara, Calif., USA). The camera is rigidly mounted on the detector of the C-arm and the transformation between the camera and CBCT origin is modeled as a rigid transformation. They are calibrated using the method mentioned herein. The tracking algorithm discussed herein is used for tracing the surgical tools.

Target localization experiment: We attached 5 radiopaque markers on a phantom and positioned the drill tip on the markers. The corresponding coordinates in the CBCT is recovered and compared by measuring the Target Registration Error (TRE). Tracking accuracy of 1.36 mm is reached when sufficient number of features are observed from the camera. An accuracy of 6.40 mm is reached when the drill is partially occluded, and 2 cm when fully occlusion (Table 9).

TABLE 9
The TRE measurements of the target localization experiment,
where δx, δy, δz, and ||δ||₂ are the Euclidean distances. Values
are reported as mean ± SD.

| | δx | δy | δz | \|\|δ\|\|₂ | |
|---|---|---|---|---|---|
| Partial | 0.02 ± 1.80 | 1.35 ± 0.85 | 5.78 ± 0.41 | 6.40 ± | 1.8 [symbol] |
| Occlusion | | | | | |
| Low | 1.28 ± 0.12 | 0.30 ± 0.19 | 1.68 ± 0.64 | 1.36 ± | 1.12 |
| Occlusion | | | | | |
| High | 17.50 ± 4.70 | 7.50 ± 2.18 | 8.91 ± 4.47 | 20.6 [symbol] ± | 4.54 |
| Occlusion | | | | | |

[symbol]

indicates data missing or illegible when filed

Tracking Accuracy: We first assessed the tracking accuracy by attaching a radiopaque marker on the drill tip and moving the drill to arbitrary poses. The tracking results are compared with measurements from the marker position in the CBCT (results shown in Table 10). The measurements show an average accuracy of 3.04 mm. Due to the symmetric geometry of the drill, the rotational element along the drill tube axis is lost under high occlusion. The best outcome is achieved when the larger portion of the handle remains visible.

TABLE 10
The measurements of the drill tracking quality,
where δx, δy, δz, and ||δ||₂ are the Euclidean
distances. Results are shown in mm.

| | CBCT | | | DEPTH | | | | ERROR | | |
|---|---|---|---|---|---|---|---|---|---|---|
| | x | y | z | x | y | z | δx | δy | δz | \|\|δ\|\|₂ |
| Pos 1 | −17.25 | −1.40 | −50.25 | −18.34 | −0.58 | −54.28 | 1.09 | 0.83 | 4.03 | 4.26 |
| Pos 2 | 12.75 | −29.76 | −8.25 | 10.30 | −25.26 | −8.90 | 2.45 | 4.50 | 0.65 | 5.16 |
| Pos 3 | −21.25 | −11.47 | −42.25 | −21.92 | −12.00 | −42.43 | 0.67 | 1.14 | 0.18 | 1.33 |
| Avg. | — | — | — | — | — | — | 1.40 | 2.16 | 1.62 | 3.04 |

To evaluate the orientational errors of the tracking method, we designed a phantom and measured the errors in a simulated surgical scenario. The phantom is comprised of a foam base and several pins which are placed at different orientations. Next, the tracked surgical drill is oriented to align with the pin's orientations. We then measure the distance from the tool tip to the desired path given by the pin's orientations, which can be easily extracted in CBCT images. The results are shown in Table 11. The measurement shows the orientation accuracy is approximately 3 mm errors even though the tool is partially occluded by the hand. However, for pin 3, since many features are occluded the tracking accuracy is worse.

TABLE 11
The measurements of the drill guidance quality. ||δ||₂ is the Euclidean
from the tool tip to the expected paths. Results are shown in mm.

| | Pin 1 | Pin 2 | Pin 3 | Pin 4 | Pin 5 |
|---|---|---|---|---|---|
| \|\|δ\|\|₂ | 3.0563 | 3.4618 | 6.3178 | 3.0304 | 2.5764 |

Discussion and Conclusion

In this work we use advanced vision-based methods to track surgical tools in C-arm guided interventions. The tracking outcome is also integrated in a mixed reality environment. Our proposed tracking system provides an alignment close to the planning solution, and supports the surgeon with the complex task of placing tools. As a consequence, it improves the efficiency in the operating room, reduces the effort and frustration, and gives the utmost confidence to the surgeon.

Moreover, using this system leads to a dramatic reduction of number of necessary X-ray images for several orthopedic interventions. With the support of this system, the surgical tools are aligned with the planning data, and thereafter a few X-ray images are required to confirm the correct placement of tools and medical instruments into the patients. Note that, this method should not be considered as a navigation system that replaces the judgment of the surgeon, but instead as a platform that gives nearly accurate 3D information to the surgeon for alignment of their instrument with the medical data.

We proposed an approach using real-time model-based tracking, and a mixed reality visualization for image-guided interventions. The visualization provides multiple simultaneous views from different perspectives. In each view, anatomical image data, the surgical site, the planned trajectory and tracked surgical tool are depicted, which facilitates intuitive perception of the target depth, orientation, and its relationship to the surgical tool, and thereby allows a faster surgical tools placement while keeping the total radiation dose to a minimum.

The RGBD camera and CBCT are registered using SAC-IA with FPFH, followed by an ICP-based refinement. The surgical tool is tracked using InSeg® with the same RGBD camera. TRE measurements are presented to assess the accuracy. The results indicate that, in general, the marker-less tracking provides reasonable accuracy of 3.04 mm. When the tracking features are fully seen by the depth camera, it can achieve an accuracy of up to 1.36 mm.

In a typical clinical scenario, the instruments are partially occluded by the hands. In this case, the approach maintains an accuracy of 6.40 mm. To further improve the tracking quality, multiple RGBD cameras could be placed to maximize the surface coverage of the tracked objection.

For several orthopedics intervention, knowledge over the correct orientation of the surgical tool (with respect to planning or medical data) is a very crucial component. We evaluated the quality of the tracking by measuring distance between tool tip position to a desired ground truth path. For the majority of cases that the drill is partially occluded, the tracking error is approximately 3 mm. This accuracy is not sufficient for precise k-wire/screw placement. However, it provides a good starting point and insertion angle that is roughly aligned with the planned trajectory. To further improve the accuracy, multiple RGBD cameras from different perspective could be integrated into the system, so that the tracking algorithm gathers sufficient information to compensate the occlusion, and therefore provides higher accuracy.

The combined visualization environment can be illustrated in a simulated procedure and demonstrates the support provided using the vision-based tracking of the surgical drill. In this setup, first, the virtual model of the drill is pre-located in the mixed reality environment (initialization). Next, the user holds the drill near the pre-defined location, in front of the camera, and attempts to nearly align it with the virtual model. Once the tracking quality reaches a certain threshold, the tracking of the surgical drill initiates. Thereafter, the surgeon uses the multi-view mixed reality scene and aligns the tool's orientation with the planned trajectory.

In conclusion, this paper presents a marker-less tracking algorithm combined with an intuitive intra-operative mixed reality visualization of the 3D medical data, surgical site, and tracked surgical tools for orthopedic interventions. This method integrates advanced computer vision techniques using RGBD cameras into a clinical setting, and enables tracking of surgical equipment in environments with high background noise and occlusion. It enables surgeons to quickly reach a better entry point for the rest of the procedure.

Although the invention has been described with reference to the above examples, it will be understood that modifications and variations are encompassed within the spirit and scope of the invention. Accordingly, the invention is limited only by the following claims.

1. A medical imaging apparatus comprising:
a) a Cone-Beam Computed Tomography (CBCT) imaging modality having an X-ray source and an X-ray detector configured to generate a series of image data for generation of a series of volumetric images, each image covering an anatomic area;
b) an auxiliary imaging modality configured to generate a series of auxiliary images; and
c) a processor having instructions to generate a global volumetric image based on the volumetric images and the auxiliary images.

2. The apparatus of claim 1, wherein the processor is configured to perform an image registration process comprising co-registering the volumetric images and the auxiliary images, wherein co-registration includes stitching of non-overlapping volumetric images to generate the global volumetric image.

3. The apparatus of claim 1, wherein the auxiliary imaging modality is an optical imaging modality configured to generate optical images.

4. The apparatus of claim 1, wherein the auxiliary imaging modality is a depth imaging modality.

5. The apparatus of claim 1, wherein the depth imaging modality is a RGB-D camera.

6. The apparatus of claim 1, wherein the imaging modalities are housed in a C-arm device.

7. The apparatus of claim 6, wherein the C-arm is configured to automatically navigate.

8. A method for generating an image, the method comprising:
a) generating a series of image data using a Cone-Beam Computed Tomography (CBCT) imaging modality for generation of a series of volumetric images, each image covering an anatomic area;
b) generating a series of auxiliary images using an auxiliary imaging modality; and
c) generating a global volumetric image based on the volumetric images and the auxiliary images, thereby generating an image.

9. The method of claim 8, wherein the global volumetric image is generated via an image registration process comprising co-registering the volumetric images and the auxiliary images, wherein co-registration includes stitching of non-overlapping volumetric images to generate the global volumetric image.

10. The method of claim 8, wherein the auxiliary imaging modality is an optical imaging modality configured to generate optical images.

11. The method of claim 8, wherein the auxiliary imaging modality is a depth imaging modality.

12. A medical robotic system comprising: a memory for receiving an image generated via the method according to claim 8; and a processor configured for at least semi-automatically controlling the medical robotic system based on the received image.

13. The medical robotic system of claim 12, wherein the received image comprises image data for a target location in a patient anatomy, and wherein controlling the medical robotic system comprises delivering a therapeutic substance or device to the target location using the medical robotic system.

14. The medical robotic system of claim 12, wherein the received image comprises image data for a target location in a patient anatomy, and wherein controlling the medical robotic system comprises applying a treatment modality to the target location using the medical robotic system.

15. Use of the medical apparatus of claim 1 to perform a medical procedure.

16. The use of claim 15, wherein the medical procedure comprises removal or insertion of a foreign body.

17. The use of claim 16, wherein the apparatus is configured to automatically track the foreign body.

18. The use of claim 17, wherein the foreign body is a wire or medical instrument.

19. The use of claim 16, wherein the foreign body is shrapnel.

20. A method of calibrating the medical apparatus of claim 1, comprising calibrating the auxiliary imaging modality to the CBCT imaging modality.

21. The method of claim 20, wherein the calibrating utilizes Fast Point Feature Histogram descriptors and a Iterative Closest Point algorithm.

22. The method according to claim 8, further comprising calibrating the auxiliary imaging modality to the CBCT imaging modality.
